(19) United States
(12) Patent Application Publication
(10) Pub. No.: US 2026/0115421 A1
(43) Pub. Date: Apr. 30, 2026

(54) LASER-CUT TUBE CATHETER ASSEMBLY HAVING VARIABLE STIFFNESS

(71) Applicant: Q'APEL MEDICAL, INC., Fremont, CA (US)

(72) Inventors: Michael TUROVSKIY, San Leandro, CA (US); Kip WETTER, San Francisco, CA (US); Jeffery KROLIK, Campbell, CA (US)

(21) Appl. No.: 19/054,675

(22) Filed: Feb. 14, 2025

Related U.S. Application Data
(60) Provisional application No. 63/714,819, filed on Oct. 31, 2024.
(60) Provisional application No. 63/753,825, filed on Feb. 4, 2025.
(60) Provisional application No. 63/758,842, filed on Feb. 14, 2025.

(51) Int. Cl. A61M 25/00

(52) U.S. Cl. CPC A61M 25/0054; A61M 25/0013; A61M 25/0045; A61M 25/0068; A61M 2205/0233; A61M 2205/32; A61M 2207/00

(57) ABSTRACT

Provided herein are methods and systems to easily vary the flexibility and/or stiffness of a catheter along its longitudinal length would be helpful in preparation and use of such catheters. Described herein are catheters comprising a catheter body comprising a first section comprising a plurality of first cuts cut; a second section adjacent to the first section, the second section comprising a plurality of second cuts; and a cut filler comprising a polymer that is filling at least one of a first cut of the plurality of the first cut and a second cut of the plurality of second cuts, wherein the first cut of the plurality of first cuts has an average first kerf that is wider than an average kerf of the second cut of the plurality of second cuts.

### CROSS-REFERENCE

This application claims the benefit of US Provisional Application No. 63/714,819, filed Oct. 31, 2024, US Provisional Application No. 63/753,825, filed Feb. 4, 2025, and US Provisional Application No. 63/758,842, filed Feb. 14, 2025, which are incorporated herein by reference in their entirety.

### BACKGROUND

The capability to vary the flexibility and/or stiffness of a catheter along its longitudinal length may be helpful in preparing a catheter tuned for a specific application requiring improved maneuverability and handleability (e.g., neurovascular application). As such, methods, and systems to easily vary the flexibility and/or stiffness of a catheter along its longitudinal length would be helpful in preparation and use of such catheters.

### SUMMARY

Catheters are often used to reach and provide a treatment in a difficult-to-reach target site of body of an individual. Depending on the anatomy and the treatment, a catheter having a tuned, variable flexibility and/or stiffness may be valuable to provide maneuverability and handleability. Such catheter may allow for reducing the time to time required to reach the target site and/or for providing a faster treatment with improved handling. In some applications, such as but not limited to acute ischemic stroke, where an artery is occluded, the ability to quickly reach the target site and provide treatment (e.g., remove occlusion, restore blood flow) may be critical to the success of the treatment The individual may have improved outcomes as measured by improved neurological and functional outcomes and/or reduced procedural complications (e.g., hemorrhage, perforation).

As such, it would be highly beneficial to be able to easily prepare a catheter having variable flexibility and/or stiffness along its longitudinal length tuned for its use. For example, a catheter having a lower stiffness and/or higher flexibility at a distal portion and a higher stiffness and/or lower flexibility at a proximal portion may allow for an improved maneuverability and handleability. Such a catheter may be beneficial in reaching an artery in the brain with a tortuous path and/or providing a treatment for neurovascular applications.

Provided herein is a catheter comprising: a catheter body comprising a first section comprising a plurality of first cuts; a second section adjacent to the first section, the second section comprising a plurality of second cuts; and a cut filler comprising a polymer that is filling at least one of a first cut of the plurality of the first cuts and a second cut of the plurality of second cuts, wherein the first cut of the plurality of first cuts has an average first kerf that is wider than an average kerf of the second cut of the plurality of second cuts; an outer jacket covering at least an outer surface of the catheter body; and a liner covering at least a portion of a lumen of the catheter body, wherein the interior surface defines a lumen of the catheter body. In some embodiments, the at least one of the plurality of first cuts and the plurality of second cuts comprise laser cuts. In some embodiments, the cut filler contacts at least one of the outer jacket or the liner. In some embodiments, the liner comprises a strike layer to facilitate bonding of the liner to the cut filler. In some embodiments, at least one of the plurality of first cuts with a plurality of first cuts is in an interrupted spiral pattern. In some embodiments, the second section is stiffer than the first section. In some embodiments, the first section is more flexible than the second section. In some embodiments, a flexibility of the first section is variable along a length of the first section. In some embodiments, a flexibility of the second section is variable along a length of the second section. In some embodiments, the plurality of first cuts are not parallel to each other. In some embodiments, the plurality of second cuts are not parallel to each other. In some embodiments, the catheter body comprises a metal. In some embodiments, the catheter body comprises a radiopaque material. In some embodiments, the plurality of first cuts has a first pitch and the plurality of the second cuts has a second pitch and wherein the first pitch is the same as the second pitch. In some embodiments, the plurality of first cuts has a first pitch and the plurality of the second cuts has a second pitch and wherein the first pitch is greater than the second pitch. In some embodiments, at least one of the average first kerf of the first cut or the average second kerf of the second cut ranges from 0.0001 inch to 0.01 inch. In some embodiments, at least one of a kerf of the first cut or a kerf of the second cut varies along a radial direction of the cut. In some embodiments, the catheter body comprises a connection joint at a distal end of the catheter body or a proximal end of the catheter body and wherein the connection joint is configured to connect the catheter to a connection joint of a second catheter. In some embodiments, the catheter comprises a flat tip at a distal end of the catheter body.

Provided herein is a method of preparing a catheter, the method comprising: (a) applying a plurality of cuts to a tube to form a cut tube having a plurality of cuts; (b) applying a first stretch to a first section of the cut tube, wherein the first stretch increases an average kerf of the plurality of cuts in the first section from a first initial kerf to a first final kerf; (c) applying a second stretch to a second section of the cut tube proximal to the first section, wherein the second stretch increases an average kerf of the plurality of cuts in the second section from a second initial kerf to a second final kerf, wherein the first final kerf is larger than the second final kerf; and (d) laminating a portion of the stretched cut tube with a polymer sheet comprising a first layer of a first polymer and a second layer of a second polymer, wherein the first polymer is more elastic than the second polymer, wherein the first polymer fills the plurality of cuts in the portion of the stretched cut tube.

### BRIEF DESCRIPTION OF THE DRAWINGS

The novel features of the technology are set forth with particularity in the appended claims. A. better understanding of the features and advantages of the present technology will be obtained by reference to the following detailed description that sets forth illustrative embodiments, in which the principles of the technology are utilized, and the accompanying drawings (also “Figure” and “FIG.” herein), of which:

FIGS. 1A-1B illustrate exemplary embodiments of a catheter

FIG. 2 illustrates an exemplary embodiment of a cut tube with an interrupted helical cut pattern.

FIGS. 3A-3D illustrate exemplary embodiments of stretched cut tube sections with varying degrees of stretch.

FIGS. 44-4B illustrate exemplary embodiments of a cut tube with a stiffer section and a more flexible section.

FIGS. 5A-5B illustrate exemplary embodiments of a catheter with an outer jacket.

### DETAILED DESCRIPTION

In order to reach and provide a treatment in a difficult-to-reach target site of a body of an individual, catheters are commonly used to reach and treat the target site Depending on the anatomy to the target site and the treatment, a catheter having a variable flexibility and/or stiffness that is tuned to provide a desired level of maneuverability and handleability may be valuable. The capability to easily prepare such a catheter having variable flexibility and/or stiffness along its longitudinal length tuned for its use would be helpful. Provided herein are variably flexible catheters and methods of preparing such catheters by cutting a tube in an interrupted pattern, applying stretch to at least a portion of the cut tube, and filling the cuts with a filler material. In some embodiments, the filler material at least partially extends through the cuts. In some embodiments, the filler material fills at least a portion of the plurality of cuts In some embodiments, the filler material sets the cuts in the stretched position in the stretched portions, thereby preparing a portion of the tube having a different mechanical property (e.g., flexibility, stiffness) from a portion that is applied with a different amount of stretch, including no stretch.

Provided herein are catheters having variable flexibility and/or stiffness along its longitudinal length and methods of preparing such catheters. In some embodiments, the catheter comprises a catheter body comprising a first section comprising a plurality of first cuts; a second section adjacent to the first section, the second section comprising a plurality of second cuts; and a cut filler comprising a polymer that is filling at least one of a first cut of the plurality of the first cut and a second cut of the plurality of second cuts. In some embodiments, the catheter further comprises a liner (also referred herein as inner liner) and an outer jacket (also referred herein as outer liner). In some embodiments, the outer jacket is in contact with the cut filler. In some embodiments, the outer jacket is bonded with the cut filler. In some embodiments, the plurality of first cuts and/or the plurality of second cuts are formed by a laser. In some embodiments, the first cut of the plurality of first cuts has an average first kerf that is wider than an average kerf of the second cut of the plurality of second cuts. In some embodiments, the first section is more flexible than the second section. In some embodiments, the second section has a higher stiffness than the first section. In some embodiments, the cut filler is difficult to handle (e.g., very soft) and may fill the cuts with an aid of an outer backing layer. In some embodiments, the outer backing layer improves handleability of the cut filler. In some embodiments, the outer backing layer allows for easier application of the cut filler than without the outer backing layer. In some embodiments, the outer jacket comprises the outer backing layer.

In some embodiments, the catheter body comprises a tube having a lumen therethrough. In some embodiments, the catheter body is configured to have a variable stiffness and/or flexibility across different portions longitudinally. In some embodiments, the catheter body provides mechanical support for the catheter. In some embodiments, the catheter body is configured to be relatively rigid along at least a portion of the catheter body. In some embodiments, the catheter body may be configured to be less rigid along at least a portion of the catheter body.

In some embodiments, the first section comprising the plurality of first cuts cut is stretched longitudinally to a first stretch ratio (e.g., ratio of first stretched distance over the unstretched distance). In some embodiments, the first stretch ratio is at least about 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio is at most about 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio ranges from about 1.01 to about 10, about 1.1 to about 5, or about 1.1 to about 2.

In some embodiments, the second section comprising the plurality of second cuts cut is stretched longitudinally to a second stretch ratio (e.g., ratio of second stretched distance over the unstretched distance). In some embodiments, the first stretch ratio is greater than the second stretch ratio. In some embodiments, the first stretch ratio is the same as the second stretch ratio. In some embodiments, no stretch is applied to the second portion. In some embodiments, the first stretch ratio is at least about 1, 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio is at most about 1, 1.01, 1.05, 1.1, 1.2, 1.3, 1.4, 1.5, 1.6, 1.7, 1.8, 1.9, 2, 3, 4, 5, 6, 7, 8, 9, or 10. In some embodiments, the first stretch ratio ranges from about 1 to about 10, about 1 to about 5, or about 1 to about 2. In some embodiments, the second stretch ratio is 1.

In some embodiments, the catheter comprises an outer jacket covering at least an outer surface of the catheter body. In some embodiments, the outer jacket comprises a polymer.

In some embodiments, the catheter comprises a liner covering at least a portion of a lumen of the catheter body. In some embodiments, the liner comprises a material having a low coefficient of friction. In some embodiments, the material is a polymer having a low coefficient of friction. Such material may help to advance one or more structures through the lumen of the catheter body. For example, the liner may comprise a polymer having a low coefficient of friction. In some embodiments, the low coefficient of friction allows a guide wire to be advanced through the central lumen with little to no resistance caused by contact between guidewire with the liner.

The catheter may be modified to have different configurations to allow for navigation through different internal tissue structures, including but not limited to arteries, veins, capillaries, organs, or tissue layers. In some embodiments, the lumen of the catheter is configured to allow the passage of a second catheter and/or a guidewire through it. In some embodiments, the second catheter and/or a guidewire may be introduced through the lumen of the catheter during a procedure. In some embodiments, the second catheter and/or a guidewire may be removed from the lumen of the catheter during a procedure. In some embodiments, one or more of the catheter bodies, the liner, and the outer jacket may be configured to achieve a desired flexibility and/or stiffness per unit of length along the catheter. In some embodiments, the second catheter may have a tapered tip. In some embodiments, the catheter provided herein may be delivered to the target site through a lumen of a sheath. In some embodiments, the catheter provided herein may fit within a lumen of a sheath.

In some embodiments, the catheter comprises a first section having a first stiffness. In some embodiments, the catheter comprises a second section having a second stiffness. In some embodiments, the second stiffness is greater than the first stiffness. In some embodiments, the first section is distal to the second section. In some embodiments, the first section is proximal to the second section. In some embodiments, the first stiffness is at least about 1%, 5%, 10%, 15%, 20%, 25%, 30%, 35%, 40%, 45%, 50%, 60%, 70%, 80%, 90%, or 100% less than the second stiffness. In some embodiments, the first stiffness is at most about 10%, 15%, 20%, 25%, 30%, 35%, 40%, 45%, 50%, 60%, 70%, 80%, 90%, 100%, 200%, 300%, 400%, 500%, 600%, 700%, 800%, 900%, or 1000% less than the second stiffness.

In some embodiments, the first section is more flexible than the second section. In some embodiments, the first flexibility is at least about 1%, 5%, 10%, 15%, 20%, 25%, 30%, 35%, 40%, 45%, 50%, 60%, 70%, 80%, 90%, or 100% greater than the second flexibility. In some embodiments, the first flexibility is at most about 10%, 15%, 20%, 25%, 30%, 35%, 40%, 45%, 50%, 60%, 70%, 80%, 90%, 100%, 200%, 300%, 400%, 500%, 600%, 700%, 800%, 900%, or 1000% greater than the second flexibility.

Described herein are methods of preparing a variable flexible catheter and such catheters. In some embodiments, a catheter comprises an inner liner, an outer jacket, and a catheter body. In some embodiments, the catheter body is positioned between the inner liner and the outer jacket. In some embodiments, the catheter body comprises a tube. In some embodiments, the catheter body comprises a plurality of cuts, thereby comprising a cut tube. In some embodiments, the plurality of cuts comprise a first plurality of cuts and a second plurality of cuts.

In some embodiments, the catheter body comprises a cut tube. The cut tube may comprise one or more cuts forming a cut pattern. The cut tube may comprise a plurality of interrupted cuts forming an interrupted spiral cut pattern along at least a portion of the tube. In some embodiments. the one or more cuts may be in the longitudinal direction, the axial direction, or a combination of thereof. In some embodiments, the shape of the cuts may be rectangular, circular, oval, oblong, elliptical, s-shaped, or the like. The cuts may be formed into any shape configured to alter one or more features of the tube or a catheter comprising the tube. In some embodiments, the tube may comprise one or more cuts configured to affect the flexibility and/or stiffness of the tube. In some embodiments, the one or more cuts extend through the thickness of the tube to form one or more cuts that provide openings, apertures, or voids. In some embodiments, the one or more cuts comprise cuts that do not extend through the thickness of the tube. For example, the cuts may etch the tube material, remove a layer of the tube material, or alter the surface of the tube. In some embodiments, the tube comprises a portion comprising one or more cuts that extend through the thickness of the tube and a portion of the tube comprising one or more cuts that do not extend through the thickness of the tube. The portion comprising the cuts extending through the tube may be more flexible than the portion comprising the cuts that do not extend through the tube.

In some embodiments, a tube comprises a plurality of cuts. In some embodiments, a cut of the plurality of cuts may be characterized at least in part by a kerf. In some embodiments, the kerf refers to the width of the cut. In some embodiments, the kerf is affected by the width of the cutting element that performed the cut. In a non-limiting example, the kerf of the cut may correspond to the width of the cutting element (e.g., blade width, laser beam width) used to cut through the material. In some embodiments, a catheter comprises one or more layers comprising a plurality of cuts. In some embodiments, at least one of the one or more layers is a tube.

In some embodiments, the kerf is defined by a distance between the edges of the cut. In some embodiments, the kerf of a cut may be affected by the amount of stretch applied to the section of the catheter body where the cut is located. In some embodiments, the kerf provides the distance in a radial direction between the edges of the cut. In some embodiments, the kerf provides the distance in an axial direction between the edges of the cut. In some embodiments, the kerf is measured along the longitudinal direction of the tube, extending from one edge of the cut to the other edge. In some embodiments, a tube comprises a plurality of cuts. In some embodiments, the plurality of cuts are not parallel to each other. In some embodiments, the plurality of cuts are parallel to each other. In some embodiments, the edges of a cut are parallel to each other. In some embodiments, the edges of a cut are not parallel to each other. In some embodiments, the edges of a cut are not parallel to each other when the section of the catheter body where the cut is located is stretched out.

In some embodiments, the cuts within the catheter body alter its mechanical properties, such as flexibility, stiffness, and/or compressibility. In some embodiments, the kerf length and width of each kerf is configured to give a catheter or a portion of a catheter a desired stiffness and/or flexibility. In some embodiments, forming the cuts in the cut tube comprises using an automated cutting mechanism. Wherein the automated cutting mechanism may be programmed to create kerfs of a specific width. In some embodiments, the automated cutting mechanism is configured to accommodate tolerance considerations where necessary

### Catheter Body

Provided herein is a catheter comprising a catheter body comprising a plurality of first cuts in a first section. In some embodiments, the catheter body comprises a plurality of second cuts in a second section. In some embodiments, the first cuts are different from the second cuts. In some embodiments, the first cuts are wider than the second cuts. In some embodiments, the first cuts are longer than the second cuts. In some embodiments, the first cuts are spaced apart further than the second cuts. In some embodiments, the first cuts are spaced the same as the second cuts. In some embodiments, the catheter body comprises a helical cut pattern. In some embodiments, a catheter body comprises a plurality of cuts, forming a cut catheter body. In some embodiments the plurality of cuts form an interrupted spiral pattern along the length of the catheter body In some embodiments, the cut catheter body comprises a plurality body segments formed by the plurality of cuts. In some embodiments, the plurality of body segments are connected by bridges formed between interrupted cuts. In some embodiments, the plurality of cuts are not parallel to each other. In some embodiments, one or more of body segments consist of a portion of the tube forming a ring around the circumference of the catheter body. In some embodiments, one or more of the body segments consist of a portion of the catheter body forming a single helical turn. In some embodiments, each of body segments consist of a portion of the tube forming a ring around the circumference of the catheter body. In some embodiments, each of body segments consists of a portion of the catheter body forming a single helix turn. In some embodiments, a body segment may comprise a height, wherein the height of the body segments body segments body segments is equal to the pitch (P) of a helical cut pattern. In some embodiments, the body segments may comprise one or more helical turns. In some embodiments, the cuts may extend completely through the wall of the catheter body. The plurality of cuts may comprise interrupted cuts such that a portion of the catheter body material is between subsequent cuts. In some embodiments, the interrupted spiral pattern may comprise a plurality of bridge elements between adjacent body segments. The bridges may connect a body segment to a proximally adjacent body segment and to a distally adjacent body segment. In some embodiments, the stiffness and/or flexibility of a cut catheter body may be varied along a length of the cut catheter body depending on the cut pattern. For example, increasing the pitch (P) between adjacent body segments can increase the stiffness of the catheter body. This may create a stiffer catheter body which may be used to create a stiffer catheter. Alternatively or in combination, reducing the pitch (P) between adjacent body segments can decrease the stiffness of the catheter body. This may make a more flexible catheter body which may be used to create a more flexible catheter. In some embodiments, the width (W) of the bridges may be increased to increase the stiffness of the catheter body. This may create a stiffer catheter body which may be used to create a stiffer catheter. In some embodiments, the width (W) of the bridges may be reduced to decrease the stiffness of the catheter body. This may make a more flexible catheter body which may be used to create a more flexible catheter. In some embodiments, the kerf (K) width of the cuts may be increased to increase the flexibility of the catheter body. This may create a more flexible catheter body which may be used to create a more flexible catheter. In some embodiments, the catheter body comprises a cut tube.

In some embodiments, the cut tube comprises a proximal and a distal uncut section. In some embodiments, the proximal and the distal uncut section are less than about 0.0020 in. In some embodiments, the proximal and the distal uncut section are more than about 0.0020 in. In some embodiments, the proximal and the distal uncut section are less than about 0.10 in. In some embodiments, the proximal and the distal uncut section are more than about 0.10 in. In some embodiments, the proximal and the distal uncut section are about 0.0001 in, about 0.0002 in, about 0.0003 in, about 0.0004 in, about 0.0005 in, about 0.0010 in, about 0.0015 in, about 0.0020 in, about 0.0030 in, about 0.0040 in, about 0.0050 in, about 0.0060 in, about 0.0070 in, about 0.0080 in, about 0.0090 in, about 0.10 in, about 0.20 in, about 0.30 in, about 0.40 in, or about 0.50 in.

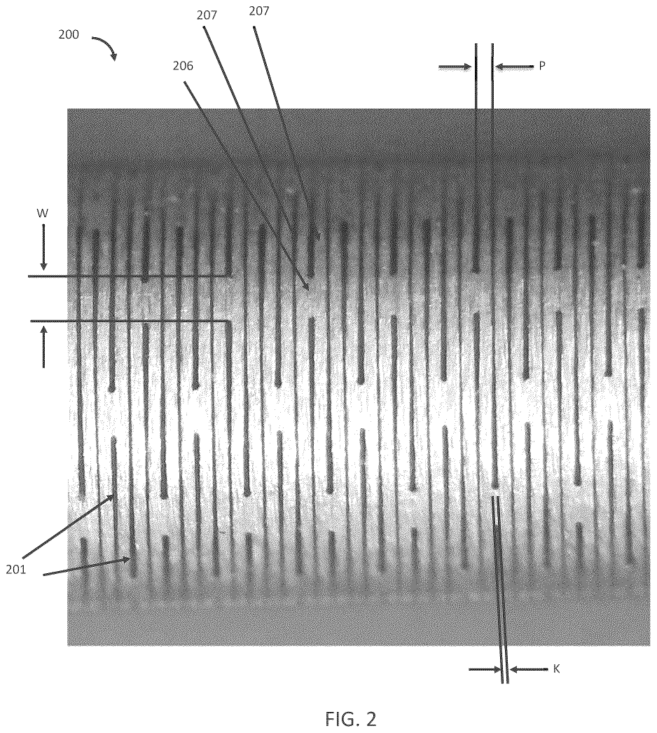

FIG. 2 illustrates an example of a cut tube 200 comprising a plurality of cuts 201 forming an interrupted spiral pattern along the length of the cut tube 200. The interrupted spiral pattern comprises a plurality of helical elements 207 formed by the cuts 201. In some embodiments, the plurality of cuts 201 are not parallel to each other. In some embodiments, one or more of helical element 207 consists of a portion of the tube forming a single helix turn. In some embodiments, each helical element 207 consists of a portion of the tube forming a single helix tum. In some embodiments, a helical element 207 may comprise a height, wherein the height of the helical element 207 is equal to the pitch (P) of said helical element 207. In some embodiments, a helical element 207 may comprise one or more helical turns. In some embodiments, the cuts 201 may extend completely through the wall of the tube 200. The plurality of cuts 201 may comprise interrupted cuts such that a portion of the tube material is between subsequent cuts. In some embodiments, the interrupted spiral pattern may comprise a plurality of bridge elements 106 between adjacent helical elements 207. The bridges 106 may connect a helical element 207 to a proximally adjacent helical element 207 and to a distally adjacent helical element 207. In some embodiments, the stiffness and/or flexibility of a cut tube may be varied along a length of the cut tube depending on the cut pattern For example, increasing the pitch (P) between adjacent helical elements 207 can increase the stiffness of the cut tube 200. This may create a stiffer tube which may be used to create a stiffer catheter. Alternatively or in combination, reducing the pitch (P) between adjacent helical elements 207 can decrease the stiffness of the tube. This may make a more flexible tube which may be used to create a more flexible catheter. In some embodiments, the width (W) of the bridges 106 may be increased to increase the stiffness of the tube. This may create a stiffer tube which may be used to create a stiffer catheter. In some embodiments, the width (W) of the bridges 106 may be reduced to decrease the stiffness of the tube. This may make a more flexible tube which may be used to create a more flexible catheter. In some embodiments, the kerf (K) width of the cuts 201 may be increased to increase the flexibility of the tube. This may create a more flexible tube which may be used to create a more flexible catheter.

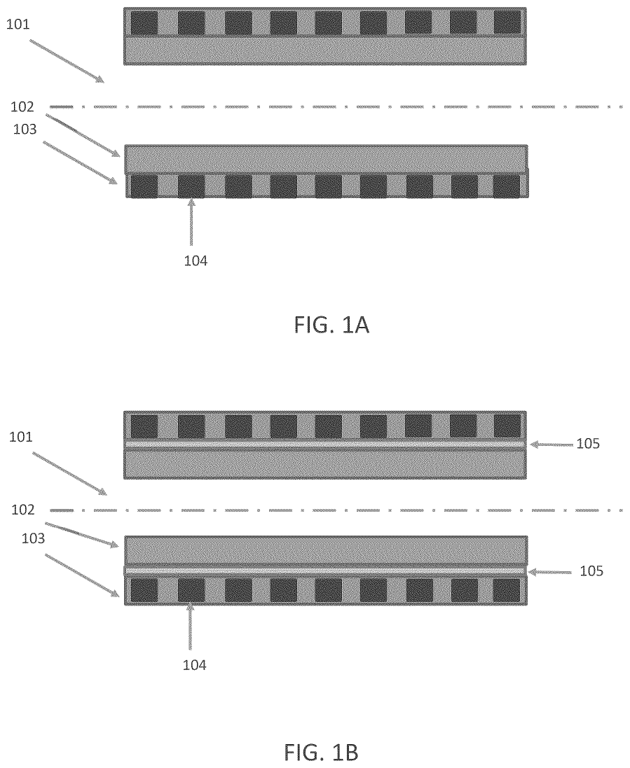

In some embodiments, the catheter body comprises a tube. In some embodiments, the catheter body comprises a cut tube. In some embodiments, the catheter body comprises a laser cut tube (LCT). In some embodiments, forming the laser cut tube comprises cutting a tube with a laser. In some embodiments, the tube is a metal hypotube. In some embodiments, the laser removes material with a characteristic width, or kerf (K). The kerf may be dependent on the laser system used, optics in the cutting beam forming, part setup when cutting or a combination thereof. In some embodiments, the cuts are formed by moving the laser along a line. This can allow for a faster cutting process wherein the laser does not need to back track or change speed during cutting. The laser may be interrupted or pulsed to form interrupted cuts. In some embodiments, a cut is formed by a single pass of the laser. In some embodiments, a kerf of a cut is substantially equal to the width of the laser. In some embodiments, a kerf that is substantially equal to the width of the laser is referred to as a laser kerf or a laser cut kerf. In some embodiments, the laser kerf is greater than 0.0015″. In some embodiments, the laser kerf is less than 0.0005″. In some embodiments, a laser kerf is about 0.0005″ to about 0.0015″. In some embodiments, the cut tube may be incorporated into a catheter wall where adjacent helical elements may function equivalent to reinforcement elements 104 of a coil support structure as illustrated in FIG. 1.

The alterations of flexibility and/or stiffness across the length of the tube may be achieved in numerous ways. In some embodiments, the cut pattern variables (pitch, kerf, cut length, pitch angle) may be varied to affect the flexibility and/or stiffness. In some embodiments, the configurations of one or more cut patterns may be varied to affect the flexibility and/or stiffness. The flexibility and/or stiffness of a tube may be configured to control the flexibility and/or stiffness of a catheter. In some embodiments, a cut tube may comprise one or more sections having different cut patterns along the length of the cut tube. The cut tube may be continuous or discontinuous along the length of the tube. In some embodiments, the cut tube may comprise 1, 2, 3, 4, 5, 6, 7, . . . , or n cut sections along the length of the tube. The cut sections may be continuous or interrupted. In some embodiments, each section of the cut tube may comprise a constant cut pattern. In some embodiments, one or more sections of the cut tube may comprise different cut patterns. In some embodiments, one or more aspects of the cut patterns may be varied e.g., the kerf or pitch. For example, each section of the cut tube may comprise a variable pitch pattern within a section. In some embodiments, a spiral-cut section may have a constant pitch. In some embodiments, the pitch may range from about 0.05 mm to about 10 mm, e.g., about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, etc. In some embodiments, the pitch may be varied within a section. In some embodiments, one or more sections comprise the same or different pitches. In some embodiments, the width or kerf of the cuts of the cut pattern may be varied along the length of the cut tube. In some embodiments, the kerf width ranges from about 0.5 microns to about 100 microns. In some embodiments, the kerf width is less than 0.5 micron. In some embodiments, the kerf width is greater than 100 microns. In some embodiments, the cut may be in a spiral pattern.

In some embodiments, each cut has a height-to-width aspect ratio. In some embodiments, the height-to-width aspect ratio is between about 1:1 to about 50:1. In some embodiments, the height-to-width aspect ratio is 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7 9:1, 8:1, 8.1:1, 8.2:1, 8.3:1, 8.4:1, 8.5:1, 8.6:1, 8.7:1, 8.8:1, 8,9:1, 9:1, 9.1:1, 9.2:1, 9.3:1, 9.4:1, 9.5:1, 9.6:1, 9.7:1, 9.8:1, 9.9:1, or 10:1. In some embodiments, the height-to-width aspect ratio is 10:1, 11:1, 12:1, 13:1, 14:1, 15:1, 16:1, 17:1, 18:1, 19:1, 20:1. In some embodiments, the height-to-width aspect ratio is 20:1, 25:1, 30:1, 35:1, 40:1, 45:1, and 50:1.

In some embodiments, the cut pattern has a cut-to-bridge ratio. In some embodiments, the cut-to-bridge ratio is between about 1:1 to about 50:1. In some embodiments, the cut-to-bridge ratio is 1:1, 1.1:1, 1.2:1, 1.3:1, 1.4:1, 1.5:1, 1.6:1, 1.7:1, 1.8:1, 1.9:1, 2:1, 2.1:1, 2 2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4.1, 6.5:1, 6.6:1, 6.7:1, 6.8:1, 6.9.1, 7:1, 7.1.1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, or 10:1. In some embodiments, the cut-to-bridge ratio is 10:1, 11:1, 12:1, 13:1, 14:1, 15:1, 16:1, 17:1, 18:1, 19:1, 20:1. In some embodiments, the cut-to-bridge ratio is 20:1, 25:1, 30:1, 35:1, 40:1, 45:1, and 50:1.

In some embodiments, an interrupted cut pattern comprises a pitch. The pitch may be measured by the edge-to-edge distance between adjacent cuts. The pitch may be adjusted to provide a desired flexibility for the catheter. In some embodiments, the pitch may range from about 0.05 mm to about 10 mm, e.g., about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, etc. In some embodiments, the pitch is less than about 0.0010 inches (in). In some embodiments, the pitch is greater than about 0.0010 in. In some embodiments, the pitch is between about 0.0010 in to about 0.050 in. In some embodiments, the pitch is between about 0.0010 in to about 0.010 in. In some embodiments, the pitch is smaller than 0.10 in. In some embodiments, the pitch is larger than 0.010 in. In some embodiments, the pitch is at least about 0.001 in, about 0.002 in, about 0.003 in, about 0.004 in, about 0.005 in, about 0.006 in, about 0.007 in, about 0.008 in, about 0.009 in, about 0.010 in, about 0.011 in, about 0,012 in, about 0,013 in, about 0.014 in, about 0.015 in, about 0.016 in, about 0.017 in, about 0.018 in, about 0.019 in, about 0.020 in, about 0.021 in, about 0.022 in, about 0.023 in, about 0.024 in, about 0.025 in, about 0.026 in, about 0.027 in, about 0.028 in, about 0.029 in, about 0.030 in about 0.031 in, about 0.032 in, about 0.033 in, about 0.034 in, about 0.035 in, about 0.036 in, about 0.037 in, about 0.038 in, about 0.039 in, about 0.040 in, about 0.041 in, about 0.042 in, about 0.043 in, about 0.044 in, about 0.045 in, about 0.046 in, about 0.047 in, about 0.048 in, about 0.049 in, or about 0.050 in. In some embodiments, the pitch is at most about 0.001 in, about 0.002 in, about 0.003 in, about 0.004 in, about 0.005 in, about 0.006 in, about 0.007 in, about 0.008 in, about 0.009 in, about 0.010 in, about 0.011 in, about 0.012 in, about 0.013 in, about 0.014 in, about 0.015 in, about 0.016 in, about 0.017 in, about 0.018 in, about 0.019 in, about 0.020 in, about 0.021 in, about 0.022 in, about 0.023 in, about 0.024 in, about 0.025 in, about 0.026 in, about 0.027 in, about 0.028 in, about 0.029 in, about 0.030 in about 0.031 in, about 0.032 in, about 0.033 in, about 0.034 in, about 0.035 in, about 0.036 in, about 0.037 in, about 0.038 in, about 0.039 in, about 0.040 in, about 0.041 in, about 0.042 in, about 0.043 in, about 0.044 in, about 0.045 in, about 0.046 in, about 0.047 in, about 0.048 in, about 0.049 in, or about 0.050 in. In some embodiments, one or more different sections of the catheter can have different pitches to vary the flexibility along the catheter length.

In some embodiments, an interrupted cut pattern may comprise a certain number of cuts for each turn or rotation. In some embodiments, there may be at least 2, 2.5, 3.5, 4, 4.5, 5, 5.5, 6, 6.5, 7, 7.5, 8, 9, or 10 cuts per turn. In some embodiments, there may be at most 2, 2.5, 3.5, 4, 4.5, 5, 5.5, 6, 6.5, 7, 7.5, 8, 9, or 10 cuts per turn. In some embodiments, there are 2, 2.5, 3.5, 4, 4.5, 5, 5.5, 6, 6.5, 7, 7.5, 8, 9, or 10 cuts per turn.

In some embodiments, the flexibility of the catheter may be altered by changing the size of the kerf. In some embodiments, the flexibility of the cut tube may be altered by changing the size of the kerf. In some embodiments, a cut tube having wider kerfs is more flexible than a cut tube having thinner kerfs.

In some embodiments, kerfs that are wider than the width of the cutting element may be desired. In some embodiments, it may be challenging to make kerfs (K) larger than the width of cutting element (e.g., laser beam) being used. While it may be possible to use laser cutting for making cuts with a kerf wider than the laser width, this may require interpolating a closed loop path for each cut. This may increase manufacturing time (and resulting part cost) as the motion control of the laser system may accelerate and decelerate for each path interpolation. In some cases, laser kerf's are too thin to provide the desired flexibility to a cut tube. In some cases, a cut tube comprising laser cut kerfs may have a limited ductility and/or flexibility. In some cases, the laser kerf of 0.0005″-0.0015″ is too thin to provide the required flexibility required for traversing tortuous anatomy. In some embodiments, the cut filler at least partially fills the cut. In some cases, laser cut kerfs are too thin to allow for the cut filler to fill in the cut. In some cases, the inadequate filling of the cut by the cut filler may result in delamination of an outer jacket and an inner liner from the cut tube, because the cut filler is unable to pass through the kerts and bond to the outer jacket and the inner liner. In some cases, the catheter having such thin cuts may not be sufficiently flexible to bend as the catheter is advanced through a tortuous anatomy.

In some cases, in order to make a cut tube more flexible, the pitch (P) of the cut pattern may be decreased and/or the widths (W) of the bridges may be decreased. In some embodiments, the pitch (P) of the cut pattern is reduced to improve flexibility of a catheter body to allow for a catheter with sufficient flexibility and/or softness to traverse through torturous anatomy. In some cases, the width (W) of the bridge may be reduced to improve flexibility of a catheter body to allow for a catheter with enough flexibility to traverse through torturous anatomy. This may allow for the highest degree of flexibility and softness allowing the catheter to be advanced through more tortuous anatomy

In some cases, in order to make a cut tube more flexible, a portion of the cut tube may be stretched. In some cases, stretching a portion of the cut tube may provide a cost-effective method of increasing the kerf width as compared to changing the cutting element or changing the cutting method. In some embodiments, stretching a cut tube will increase the width of the cut kerfs. This may increase the flexibility and/or decrease the stiffness of the cut tube. In some embodiments, stretching a cut tube to increase the width of the cuts may provide a more time efficient and cost-effective method for increasing kerf width and producing a more flexible catheter. In some embodiments, a stretched cut tube may comprise varying degrees of stretch along the length of the cut tube. In some embodiments, the degree of stretch of the stretched cut tube may increase along the length of the cut tube. In some embodiments, the distal end of the cut tube has a higher degree of stretch than the proximal end of the cut tube. In some embodiments the proximal end of a stretched cut tube has no stretch. In some embodiments the proximal end of a stretched cut tube has a minimal degree of stretch such that the size of the kerfs at the proximal end and/or the flexibility of the proximal does not change.

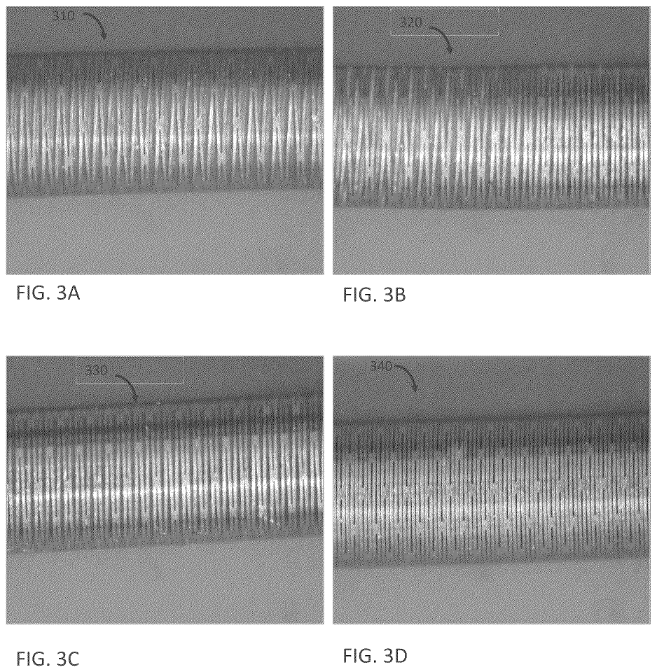

FIGS. 3A-3D illustrate an example of a stretched cut tube having varying degrees of stretch. In some embodiments, the degree of stretch increases along the length of the cut tube. FIG. 3A illustrates a distal portion 300 of a cut tube. The distal portion 300 may have the greatest degree of stretch along the length of the cut tube. FIGS. 3B-3C illustrate intermediate sections of the cut tube with decreasing degrees of stretch, wherein cut tube section 310 may be distal to cut tube section 320. FIG. 3D illustrates a most proximal section 330 of the cut tube. In some embodiments, the most proximal section of a cut tube may have the least degree of stretch. In some embodiments, the proximal section of the cut tube 330 may not be stretched. It can be appreciated in FIG. 3A-3D that varying degrees of stretch may be applied to modify both the bonding between the outer jacket and the liner as well as the catheter stiffness.

In some embodiments, a cut tube comprising a plurality of cuts is configured to be stretched, wherein one or more of the plurality of cuts changes from a first kerf width to a second kerf width. Stretching the cut tube may cause a center width of the cuts (and kerfs) of the cut tube to increase. For example, stretching the cut tube causes a distance of the center of a cut to be wider than the width of the first kerf width formed by the cut. In some embodiments, the stretched cuts may be widest at a center of the cut and narrowest at the ends of the cut. In some embodiments, the cuts narrow from a widest point at the center of the cut to a narrowest point at each end of the cut. In some embodiments, the width at the narrowest point of the cut is substantially equal to the initial kerf width. In some embodiments, one or more stretched cuts may have a widest point at the end of the cut and a narrowest point at the middle of the cut. In some embodiments, one or more stretched cuts may form an hourglass shape. In some embodiments, one or more stretched cuts may have a consistent width along the length of the cut.

In some embodiments, a first kerf width is stretched longitudinally to a second kerf width. In some embodiments, the second kerf width is stretched to at least about 1.01x, 1.05x, 1.1x, 1.2x, 1.3x, 1.4x, 1.5x, 1.6x, 1.7x, 1.8x, 1.9x, 2x, 3x, 4x, 5x, 6x, 7x, 8x, 9x, or 10x. In some embodiments, the second kerf width is stretched to at most about 1.01x, 1.05x, 1. 1x, 1.2x, 1.3x, 1.4x, 1.5x, 1.6x, 1.7x, 1.8x, 1.9x, 2x, 3x, 4x, 5x, 6x, 7x, 8x, 9x, or 10x. In some embodiments, the second kerf width is between about 1.01x to about 10x the first cut width. In some embodiments, the second kerf width is between about 1.1x to about 5x the first kerf width. In some embodiments, the second kerf width is between about 1.1x to about 2x the first kerf width.

In some embodiments, after a cut tube has been stretched to open one or more kerfs, an outer layer may be laminated to an inner layer of the catheter. This may help preserve the stretched configuration of the cut tube. In some embodiments, the outer layer comprises an outer jacket of the catheter. In some embodiments, the inner layer comprises a catheter liner. In some embodiments the inner layer comprises a strike layer In some embodiments, laminating the outer layer of the catheter to the inner layer during assembly holds the structure and configuration of a stretched cut tube. This may allow the stretched cut tube to maintain the wider (stretched) kerf widths after assembly. In some embodiments, stretching the cut tube to open the cuts may improve lamination between an outer layer and inner layer of the catheter. For example, a distal section of the cut tube having the greatest degree of stretch exhibits the best lamination between a liner and outer jacket of the catheter because the wider kerfs provide more contact between the outer and inner layers sandwiching the cut tube. In some embodiments, the portion of the catheter comprising the most stretched portion of the cut tube exhibits excellent lamination between outer jacket and the liner and is also more flexible than a catheter built from the same materials but not stretched. In some embodiments, varying degrees of stretch may be applied to modify both the bonding between the outer jacket and the liner as well as the catheter stiffness. In some embodiments, the degree of stretch of the cut tube gets higher toward the distal end of the catheter. In some embodiments, the outer layer may comprise a cut filler material. In some embodiments, the cut filler material is on an inner facing surface of the outer jacket. In some embodiments, the cut filler material is configured to fill in the kerfs of the cut tube. In some embodiments, the cut filler at least partially fills the cut.

Described herein are methods, devices, and systems configured for variable flexibility and/or stiffness of catheters. In some embodiments, a catheter is configured to have variable stiffness and/or flexibility along the length of the catheter. In some embodiments, the variable stiffness catheter comprises a tube extending at least part of the length of the catheter. In some embodiments, the variable stiffness catheter comprises a tube extending the full length of the catheter or substantially the full length of the catheter. In some embodiments, the tube is configured to affect the flexibility and/or stiffness of the variable stiffness catheter. In some embodiments, the tube is a cut tube. In some embodiments, the variable stiffness catheter comprises a cut tube configured to have a variable stiffness along the length of the cut tube. In some embodiments, the stiffness and/or flexibility of the cut tube is varied by varying the kerf width of the cuts along the length of the cut tube. In some embodiments, at least a portion of the cut tube is stretched, wherein stretching the cut tube alters the stiffness and/or flexibility of the cut tube, wherein stretching the cut tube increases the flexibility of the cut tube. In some embodiments, the entire length of the cut tube is stretched. In some embodiments, a cut tube comprises a first stretched portion configured to have a first stiffness and flexibility. In some additional embodiments, the cut tube may comprise one or more additional stretched portions. The one or more additional stretched portion may be configured to have flexibility and/or stiffness that is different than the first portion. In some embodiments, the one or more additional stretched portions may comprise one or more stretched portions having a stiffness and/or flexibility that is the same as the first stretched portion. In some embodiments, a cut tube comprises two or more stretched portions where in each portion of the cut tube is configured to have a different stiffness and flexibility. In some embodiments, a cut tube comprises a plurality of stretched portions wherein the stiffness and flexibility alternate between adjacent portions. In some embodiments, a cut tube may comprise a distal stretched portion. The distal stretched portion may be more flexible than a proximal portion of the cut tube. In some embodiments, the cut tube may be more flexible at a distal end of the cut tube than a proximal end of the cut tube. In some embodiments, the flexibility of the cut tube may increase at a consistent rate from the proximal end of the cut tube to a distal end of the cut tube. In some embodiments, the flexibility of the cut tube may increase at a variable rate from approximately the cut tube to a distal end of the cut tube. In some embodiments, the rate of change in the flexibility increases as you move distally along the length of the cut tube. In some embodiments, the rate of change in the flexibility decreases as you move distally along the length of the cut tube. In some embodiments, by stretching the cut tube, the size of the kerf is increased (or opened) such that the kerf width is greater than the original kerf width. In some embodiments, the flexibility and/or stiffness of the cut tube is based at least in part on how far the cut tube is stretched.

In some embodiments, heat is applied to the cut tube. In some embodiments, heat is applied to the cut and stretched tube. In some embodiments, a cut tube is stretched and then heated in the stretched configuration. In some embodiments, the application of heat aids in heat setting the cut tube. In some embodiments, a stretched cut tube may be set in place in the stretched configuration by the application of heat without applying a stretch. In some embodiments, a stretched cut tube may keep its stretched configuration after the application of heat without applying a stretch. In some embodiments, heat setting of a cut tube in the stretched configuration may make the assembly of the catheter easier. In some embodiments, the heat setting may make application of the kerf filler, outer jacket, and/or inner liner easier than without the heat setting by keeping the cut tube in the stretched configuration without any stretching applied. In some embodiments, heat setting of a cut tube in the stretched configuration may reduce or eliminate the elastic energy stored in the stretched cut tube. In some embodiments, reducing or eliminating elastic energy may improve mechanical stability of the catheter. In some embodiments, reducing or eliminating elastic energy may facilitate preparation of the catheter. In some embodiments, reducing or eliminating elastic energy may make it easier to handle the cut tube. In some embodiments, heat setting reduces work hardening caused by plastic deformation. In some embodiments, heat setting improves mechanical stability of the catheter.

In some embodiments, a cut tube is heated and then cooled. In some embodiments, a cut tube is heated and then cooled rapidly. In some embodiments, cooling is by an air cool or water quench or a combination thereof.

In some embodiments, heat is applied to the cut tube for at least about 0.1, 0.5, 1, 5, 10, 15, 20, 25, 30, 35, 40, 45, 50, 55, or 60 minutes. In some embodiments, heat is applied to the cut tube for at most about 1, 5, 10, 15, 20, 25, 30, 35, 40, 45, 50, 55, 60, 70, 80, 90, 100, 110, or 120 minutes. In some embodiments, heat is applied to the cut tube for ranges from about 0.5 to about 120 minutes, about 1 to about 60 minutes, about 1 to about 45 minutes, about 1 to about 30 minutes, about 1 to about 20 minutes, or about 1 to about 10 minutes.

In some embodiments, heat applied to the cut tube is at least about 100° C., 200° C., 300° C., 400° C., 500° C., 600° C., 700° C., 800° C., 900° C., 1000° C., 1100° C., 1200° C., 1300° C., 1400° C., or 1500° C.. In some embodiments, heat applied to the cut tube is at most about 100° C., 200° C., 300° C., 400° C., 500° C., 600° C., 700° C., 800° C., 900° C., 1000° C., 1100° C., 1200° C., 1300° C., 1400° C., or 1500° C. In some embodiments, heat applied to the cut tube ranges from about 100° C. to about 1500° C., about 100° C. to about 1000° C., about 200° C. to about 800° C., about 300° C. to about 700° C., about 500° C. to about 1500° C., about 700° C. to about 1500° C., or about 800° C. to about 1300° C.

In some embodiments, the cut tube is heat to at least about 100° C., 200° C., 300° C., 400° C., 500° C., 600° C., 700° C., 800° C., 900° C., 1000° C., 1100° C., 1200° C., 1300° C., 1400° C., or 1500° C. In some embodiments, the cut tube is heat to at most about 100° C., 200° C., 300° C., 400° C., 500° C., 600° C., 700° C., 800° C., 900° C., 1000° C., 1100° C., 1200° C., 1300° C., 1400° C., or 1500° C. In some embodiments, the cut tube is heat to a range of about 100° C. to about 1500° C., about 100° C. to about 1000° C., about 200° C. to about 800° C., about 300° C. to about 700° C., about 500° C. to about 1500° C., about 700° C. to about 1500° C., or about 800° C. to about 1300° C.

In some embodiments, a cut tube comprising NiTi can be heat set. In some embodiments, a cut tube comprising NiTi maintains its superelastic property after heat setting. In some embodiments, a cut tube comprising NiTi that is heat set can bend when a load is applied. In some embodiments, a cut tube comprising NiTi may be stretched to deformations within its superelastic plateau. In some embodiments, a cut tube comprising NiTi is heat set after being stretched to deformations within its superelastic plateau. In some embodiments, a cut tube comprising NiTi may be heat set at a temperature range of about 400° C. to about 600° C. for 1 to30 minutes followed by a rapid temperature drop (e.g., a quench). In some embodiments, a cut tube comprising NiTi may be heat set at a temperature of about 500° C. for 1 to 30 minutes followed by a rapid temperature drop (e.g., a quench). In some embodiments, a cut tube comprising NiTi may be heat set at a temperature range of about 400° C. to about 600° C. In some embodiments, a cut tube comprising NiTi may be heat set for 1 to 30 minutes.

In some embodiments, a cut tube comprising stainless steel may be heat set at a temperature of about 900° C. to about 1300° C. for 1 to 60 minutes and then cooled. In some embodiments, a cut tube comprising stainless steel may be heat set at a temperature of about 1040° C. to about 1175° C. for 1 to 30 minutes and then cooled.

In some embodiments, the catheter comprises a tip at a distal end of the catheter body. In some embodiments, the tip is a flat tip. In some embodiments, the flat tip comprises a flat distal end. In some embodiments, the tip comprises one or more distal extensions. In some embodiments, the distal extension is flexible. In some embodiments, the tip does not have any distal extensions. In some embodiments, the tip comprises one or more holes. In some embodiments, the tip is flexible. In some embodiments, the tip is made of a flexible material. In some embodiments, the holes aids in providing flexibility to the tip.

### Cutting Methods

Methods for forming a cut tube as described herein may comprise one or more methods for mechanically cutting a pattern into a tube. In some embodiments, cutting the pattern into the tube comprises one or more non-mechanical cutting methods. In some embodiments, the notches, holes or cuts may be formed in the tube using a laser (solid-state, femtosecond laser, or YAG laser, for example), electrical discharge (electrical discharge machining (EDM)), plasma blade, pressurized fluid jet (waterjet), a lathe, a saw, a blade, chemical etching, photo-etching, or a combination thereof. In some embodiments, methods of cutting a pattern into a tube may comprise laser cutting, plasma cutting, hydrocutting, thermal cutting, mechanical cutting, or a combination thereof.

In some embodiments, a tube comprises one material. In some embodiments, the tube comprises two or more materials. In some embodiments, the tube comprises a metal, metal alloy, polymer, or combination thereof. The tube may comprise nitinol, stainless steel, or a combination thereof. In some embodiments, the tube comprises stainless steel, nickel titanium (nitinol), or polymers. In some embodiments, the tube comprises, super elastic NiTi alloy super elastic nickel titanium, shape memory nickel titanium, TiNi, nickel titanium, NiTIHE, NiTiPd, NiMnGa, stainless steel. In some embodiments, the stainless steel may comprise Stainless having an SAE grade in the 200, 300, or 400 series, precipitation hardened (PH) stainless steel, other spring steel or other high tensile strength material or other biocompatible metal material. In some embodiments, the material of a tube comprises superelastic or shape memory metal (e.g., nickel titanium), Alternatively or in combination, the material of a tube comprises stainless steel.

### Catheter Layers

In some embodiments, the one or more layers of the outer jacket may comprise an inner layer and an outer layer. In some embodiments, the liner comprises one or more layers. In some embodiments, the liner comprises a strike layer. In some embodiments, the multiple layers of the outer jacket may allow the inner layer to be incorporated in between the catheter body (such as LCT) and the outer layer In some embodiments, the outer layer may be configured to lie on top of the inner layer. Alternatively, or in combination, the outer layer may be configured to lie on top of the catheter body. In some embodiments, the inner layer comprises a first polymer. In some embodiments, the outer material comprises a second polymer. In some embodiments, the second polymer is configured to make the first polymer easier to handle. In some embodiments, the inner material may comprise a soft tacky material. In some embodiments, the inner layer may comprise a cut filler material also referred to as a filler material, configured to fill the voids (e.g., kerfs) of the catheter body. In some embodiments, the filler material is configured to fill the kerf's of a cut catheter body. In some embodiments, the filler material is configured to elongate and compress while the catheter is stretched. In some embodiments, using a softer, more elastic filler material will result in a more flexible catheter. In some embodiments, the outer layer of the outer jacket may be configured to be harder (i.e., more rigid or less flexible) than the filler material. In some embodiments, the material of the outer layer may be configured to improve tensile strength of the catheter, reduce friction properties of the outer layer, and/or improve compatibility with one or more coatings applied to the catheter. In some embodiments, having a multiple layer catheter makes catheter assembly easier, because the more flexible materials of the inner layer may be very tacky and/or difficult to handle. In some embodiments, the filler material may comprise a non-flowing soft material. In some embodiments, the inner layer comprises a gel material or rubber material. In some embodiments, the harder outer layer may be configured to make the inner layer easier to handle and load over the catheter body and liner assembly. In some embodiments, the filler material may contact the outer jacket. In some embodiments, an outer layer is configured to be laminated to the catheter body. In some embodiments, an outer layer is configured to be laminated to the liner. In some embodiments, an outer layer is configured to be laminated in combination with the inner layer.

In some embodiments, the outer layer of the catheter may be formed of a stiffer material than the inner layer. In some embodiments, the outer layer material may be configured to provide improved tensile strength of the catheter assembly, reduce friction properties of the outer layer, improve compatibility with any coatings applied to the catheter, and/or a combination thereof. In some embodiments, an outer layer is configured to be laminated to the catheter assembly in combination with the inner layer. In some embodiments, a stiffer outer layer is configured to make catheter assembly easier, because the very soft materials of the inner layer may be very tacky or otherwise difficult to handle. In some embodiments, the stiffer outer layer may make the tube easier to handle and load over the LCT and liner assembly.

In some embodiments, the filler material is configured to fill in the space of the cuts. In some embodiments, the filler material is a viscous liquid. In some embodiments, the filler material comprises a non-Newtonian fluid. In some embodiments, the filler material is bonded to the outer jacket. In some embodiments, the filler material contacts the liner. In some embodiments, the filler material is bonded to the liner. In some embodiments, the liner comprises a strike layer configured to facilitate bonding of the liner to the filler material. In some embodiments, a surface of the liner is modified to facilitate bonding between the liner and the filler material.

In some embodiments, the inner layer of the outer jacket comprises a material having a durometer of at least 0.1 Shore 00, 0.5 Shore 00, 1.0 Shore 00, 2.0 Shore 00, 3.0 Shore 00, 4.0 Shore 00, 5.0 Shore 00, 6.0 Shore 00, 7.0 Shore 00, 8.0 Shore 00, 9.0 Shore 00, 10 Shore 00, 20 Shore 00, 30 Shore 00, 40 Shore 00, 50 Shore 00, 60 Shore 00, 70 Shore 00, 80 Shore 00, 90 Shore 00, 0.1 Shore A, 0.5 Shore A, 1.0 Shore A, 2.0 Shore A, 3.0 Shore A, 4.0 Shore A, 5.0 Shore A, 6.0 Shore A, 7.0 Shore A, 8.0 Shore A, 9.0 Shore A, 10 Shore A, 11 Shore A, 12 Shore A, 13 Shore A, 14 Shore A, 15 Shore A, 16 Shore A, 17 Shore A, 18 Shore A, 19 Shore A, 20 Shore A, 21 Shore A, 22 Shore A, 23 Shore A, 24 Shore A, 25 Shore A, 26 Shore A, 27 Shore A, 28 Shore A, 29 Shore A, 30 Shore A, 31 Shore A, 32 Shore A, 33 Shore A, 34 Shore A, 35 Shore A, 36 Shore A, 37 Shore A, 38 Shore A, 39 Shore A, 40 Shore A, 41 Shore A, 42 Shore A, 43 Shore A, 44 Shore A. 45 Shore A, 46 Shore A, 47 Shore A, 48 Shore A, 49 Shore A, 50 Shore A, 51 Shore A, 52 Shore A, 53 Shore A, 54 Shore A, 55 Shore A, 56 Shore A, 57 Shore A, 58 Shore A, 59 Shore A, 60 Shore A, 61 Shore A, 62 Shore A, 63 Shore A, 64 Shore A, 65 Shore A, 66 Shore A, 67 Shore A, 68 Shore A, 69 Shore A, 70 Shore A, 71 Shore A, 72 Shore A, 73 Shore A, 74 Shore A, 75 Shore A, 76 Shore A, 77 Shore A, 78 Shore A, 79 Shore A, 80 Shore A, 81 Shore A, 82 Shore A, 83 Shore A, 84 Shore A, 85 Shore A, 86 Shore A, 87 Shore A, 88 Shore A, 89 Shore A, 90 Shore A. In some embodiments, the inner layer of the outer jacket comprises a material having a durometer of at most 0.1 Shore 00, 0.5 Shore 00, 1.0 Shore 00, 2.0 Shore 00, 3.0 Shore 00, 4.0 Shore 00, 5.0 Shore 00, 6.0 Shore 00, 7.0 Shore 00, 8.0 Shore 00, 9.0 Shore 00, 10 Shore 00, 20 Shore 00, 30 Shore 00, 40 Shore 00, 50 Shore 00, 60 Shore 00, 70 Shore 00, 80 Shore 00, 90 Shore 00, 0.1 Shore A, 0.5 Shore A, 1.0 Shore A, 2.0 Shore A, 3.0 Shore A, 4.0 Shore A, 5.0 Shore A, 6.0 Shore A, 7.0 Shore A, 8.0 Shore A, 9.0 Shore A, 10 Shore A, 11 Shore A, 12 Shore A, 13 Shore A, 14 Shore A, 15 Shore A, 16 Shore A, 17 Shore A, 18 Shore A, 19 Shore A, 20 Shore A, 21 Shore A, 22 Shore A, 23 Shore A, 24 Shore A, 25 Shore A, 26 Shore A, 27 Shore A, 28 Shore A, 29 Shore A, 30 Shore A, 31 Shore A, 32 Shore A, 33 Shore A, 34 Shore A, 35 Shore A, 36 Shore A, 37 Shore A, 38 Shore A, 39 Shore A, 40 Shore A, 41 Shore A, 42 Shore A, 43 Shore A, 44 Shore A, 45 Shore A, 46 Shore A, 47 Shore A, 48 Shore A, 49 Shore A, 50 Shore A, 51 Shore A, 52 Shore A, 53 Shore A, 54 Shore A, 55 Shore A, 56 Shore A, 57 Shore A, 58 Shore A, 59 Shore A, 60 Shore A, 61 Shore A, 62 Shore A, 63 Shore A, 64 Shore A, 65 Shore A, 66 Shore A, 67 Shore A, 68 Shore A, 69 Shore A, 70 Shore A, 71 Shore A, 72 Shore A, 73 Shore A, 74 Shore A, 75 Shore A, 76 Shore A, 77 Shore A, 78 Shore A, 79 Shore A, 80 Shore A, 81 Shore A, 82 Shore A, 83 Shore A, 84 Shore A, 85 Shore A, 86 Shore A, 87 Shore A, 88 Shore A, 89 Shore A, 90 Shore A.

In some embodiments, a catheter as described herein comprises a catheter body, wherein the catheter body is formed of composite structures that have different elements with specific functions to optimize catheter performance. As illustrated in FIG. 1, a catheter may comprise an inner layer 102, and outer layer 103 (or outer jacket), and a catheter body 104. The inner-most layer 102 of the catheter (meaning, the layer adjacent to the central lumen 101), may be a liner. In some embodiments, the liner 102 may comprise a polymer having a low coefficient of friction. The liner material may comprise pebax (friction modified or natural), PTFE, or PE. In some embodiments, a liner material may be selected based on a coefficient of friction with other items that may be tracked through the central lumen of the catheter and/or bondability to an outer jacket 103. In some embodiments, a liner 102 may comprise one or more surface modification. In some embodiments, the surface modifications of the liner 102 may comprise one or more of a chemical modification, textural modification, or plasma modification. In some embodiments, the surface modification may be configured to improve the bondability of the liner 102 to the jacket material 103. In some embodiments, a catheter may further comprise strike layer 105, as illustrated in FIG. 1B. In some embodiments, the strike layer 105 is configured to improve bondability between the liner 102 and jacket 103. In some embodiments, the strike layer 105, may be a strike layer 105. In some embodiments, the strike layer is configured to improve bondability between the liner and jacket.

In some embodiments, the catheter comprises one or more layers similar to the catheter shown in FIG. 1. In some embodiments, having a multiple layer catheter as shown in FIGS. 5A and 5B, makes catheter assembly easier, because the more flexible materials of the inner layer may be very tacky and/or difficult to handle. FIGS. 5A and 5B illustrate an embodiment of a catheter comprising an inner liner 502, a strike layer 503, a catheter body 504, and a multi-layer outer jacket 503. In some embodiments, catheter body comprises an outer jacket 503. In some embodiments, the outer jacket 503 comprises an inner layer 503a and an outer layer 503b. FIG. SA illustrates an example of the multilayer catheter cross-section comprising an inner liner 502, a strike layer 503, a catheter body 504, and a multi-layer outer jacket 503, wherein the layers are assembled, but not yet laminated together. FIG. SB illustrates the multilayer catheter cross-section comprising the layers of the catheter illustrated in FIG. 5A, after lamination In some embodiments, the catheter comprises a liner 502. In some embodiments, the liner 502 further comprises a strike layer 505. In some embodiments, the multiple layers of the outer jacket 503 may allow the inner layer 503a to be incorporated in between the catheter body 504 (such as LCT) and the outer layer 503b. In some embodiments, the outer layer 503b may be configured to lie on top of the catheter body 504. In some embodiments, the inner layer 503a may comprise a cut filler material also referred to as a filler material, configured to fill the voids (e.g., kerfs) of the catheter body. In some embodiments, the filler material is configured to fill the kerf's of the catheter body. In some embodiments, the filler material is configured to elongate and compress while the catheter is stretched. In some embodiments, using a softer, more elastic filler material will result in a more flexible catheter. In some embodiments, the outer layer 503b of the outer jacket 503 may be configured to be harder (i.e., more rigid or less flexible) than the filler material. In some embodiments, the material of the outer layer 503b may be configured to improve tensile strength of the catheter, reduce friction properties of the outer layer, and/or improve compatibility with one or more coatings applied to the catheter. In some embodiments, having a multiple layer tube as shown in FIGS. 5A-B may make catheter assembly easier, because the more flexible materials of the inner layer 503a may be very tacky and/or difficult to handle. In some embodiments, the harder outer layer 503b may be configured to make the inner layer 503a easier to handle and load over the catheter body and liner assembly. In some embodiments, the filler material may contact the outer jacket.

In some embodiments, the outer layer 503b of the catheter, may be formed of a stiffer material than the inner layer 503a. In some embodiments, the outer layer 503b material may be configured to provide improved tensile strength of the catheter assembly, reduce friction properties of the outer layer, improve compatibility with any coatings applied to the catheter, and/or a combination thereof. In some embodiments, an outer layer 503b is configured to be laminated to the catheter assembly in combination with the inner layer 503a. In some embodiments, a stiffer outer layer 503b is configured to make catheter assembly easier, because the very soft materials of the inner layer 503a may be very tacky or otherwise difficult to handle. In some embodiments, the stiffer outer layer 503b may make the tube 503 easier to handle and load over the LCT and liner 502 assembly.

In some embodiments, the filler material is configured to fill in the space of the laser cuts. In some embodiments, the filler material is a viscous liquid. In some embodiments, the filler material comprises a non-Newtonian fluid. In some embodiments, the filler material 503a is bonded to the outer jacket. 503 In some embodiments, the filler material 503a contacts the liner 502. In some embodiments, the filler material 503a is bonded to the liner 502. In some embodiments, the liner 502 comprises a strike layer 505 configured to facilitate bonding of the liner 502 to the filler material 503a. In some embodiments, a surface of the liner 502 is modified to facilitate bonding between the liner 502 and the filler material 503a.

In some embodiments, a catheter as described herein comprises a catheter body, wherein the catheter body is formed of composite structures that have different elements with specific functions to optimize catheter performance. In some embodiments, a catheter may comprise an inner layer, an outer layer (or outer jacket), and a catheter body. The inner-most layer of the catheter (meaning, the layer adjacent to the central lumen), may be a liner.

In some embodiments, the liner may comprise a polymer having a low coefficient of friction. The liner material may comprise pebax (friction modified or natural), PTFE, or PE. In some embodiments, a liner material may be selected based on a coefficient of friction with other items that may be tracked through the central lumen of the catheter and/or bondability to an outer jacket. In some embodiments, a liner may comprise one or more surface modifications. In some embodiments, the surface modifications of the liner may comprise one or more of a chemical modification, textural modification, or plasma modification. In some embodiments, the surface modification may be configured to improve the bondability of the liner to the jacket material. In some embodiments, a catheter may further comprise strike layer. In some embodiments, the strike layer is configured to improve bondability between the liner and jacket. In some embodiments, the strike layer, may be a strike layer. In some embodiments, the strike layer is configured to improve bondability between the liner and jacket In some embodiments, the strike layer has a thickness of at least about 0.0001 inches. In some embodiments, the strike layer has a thickness of at most about 0.003 inches. In some embodiments, the strike layer has a thickness in the range of about 0.0001 inches to about 0.003 inches. In some embodiments, the strike layer may comprise a polymer that exhibits good bondability to the jacket material In some embodiments, the strike layer is provided over the liner prior to the catheter. This may provide more assurance that a good bond is established between the liner and jacket. In some embodiments, a catheter comprises a catheter body. The catheter body may be configured to improve the kink resistance of the catheter wall. In some embodiments, the catheter body may be metallic (e.g., stainless steel, superelastic NiTi, or radiopaque materials such as tungsten or platinum/iridium alloy). In some embodiments, the catheter body may be a polymeric material, including but not limited to LCP (liquid crystal polymer), polyimide, PEEK, or nylon, or a combination thereof. The catheter body may take the form of a tubular braid or a coil.

In some embodiments, the catheter body comprises a plurality of cuts. In some embodiments, the plurality of cuts form a cut pattern along at least a portion of the catheter body. In some embodiment, the cut pattern may be a continuous cut pattern. In some embodiments, the cut pattern may be an interrupted cut pattern. In some embodiments, the cut pattern may comprise one or more different cut patterns along one or more portions of the catheter body. In some embodiments, the cut pattern comprises one or more of longitudinal cuts, axial cuts, diagonal cuts, curved cuts, or any other shaped cut. In some embodiments, the plurality of cuts form an interrupted spiral pattern In some embodiments, the catheter body may provide an improvement over existing reinforcement elements like a coil or braid. The catheter body may be made from the same materials as the traditional reinforcement element materials. In some embodiments, to create the cut pattern of the catheter body, a cutting element is passed over the tube in a helical path. In some embodiments, the cutting element cuts completely through the wall of the catheter body In some embodiments the cutting element cuts a helical path, wherein the cut catheter body would resemble a coil. In some embodiments, the cutting element is a laser, a plasma blade, a pressurized fluid jet (waterjet), a lathe, a saw, a blade, or any other suitable cutting element.

In some embodiments, the catheter body comprises a cut tube. In some embodiments, the cut tube as described herein, may provide an improvement over existing reinforcement elements like a coil or braid. The tube may be made from the same materials as the traditional reinforcement element materials. In some embodiments, to create the interrupted spiral pattern of the cut tube, a laser is passed over the tube in a helical path. In some embodiments, the laser cuts completely through the wall of the tube support material and cuts the entire helical path, wherein the cut tube would be a structure resembling a coil. In some embodiments, to create the cut pattern of the cut tube, a cutting element is passed over the tube in a helical path. In some embodiments, the cutting element cuts completely through the wall of the cut tube material. In some embodiments the cutting element cuts a helical path, wherein the cut tube would resemble a coil. In some embodiments, the cutting element is a laser, a plasma blade, a pressurized fluid jet (waterjet), a lathe, a saw, a blade, or any other suitable cutting element.

In some embodiments, a catheter comprises at least, an inner liner layer, a catheter body, and an outer jacket layer. In some embodiments, the catheter body comprises a tube. In some embodiments, the liner is bonded to an inner surface of the catheter body. In some embodiments, the liner is bonded to an inner surface of the tube. In some embodiments, the inner surface may be a flat surface. In some embodiments, the outer jacket layer may comprise a polymeric material, including but not limited to HDPE, PTFE, ETFE, FEP, Pebax, nylon, or other polymer material, or combination thereof. In some embodiments, the outer jacket is fused over the catheter body.

In some embodiments, at least a portion of the lumen of the catheter body comprises a polymer liner bonded to the inner wall of the catheter body. In some embodiments, the polymer liner may form a tube. In some embodiments, the catheter body may be configured co-axially within the lumen of the catheter body. In some embodiments, the liner may include at least two polymer layers. In some embodiments, the liner is bonded to the inner wall of the catheter body along the length of the catheter body. In some embodiments, the liner is bonded continuously to the inner wall of the catheter body along the entire length of the catheter body. In some embodiments, the liner is bonded to two or more sections of the inner wall of the catheter body spaced apart along the longitudinal axis of the catheter body. The polymer liner may be bonded to the inner wall of the catheter body in a continuous helical pattern running along at least a portion of the length of the catheter body. The polymer liner may be bonded to the inner wall of the catheter body by melting the polymer to the catheter body at one or more sections of the catheter body. In some embodiments, the surface of the liner facing the lumen is coated with a lubricous material.

In some embodiments, the catheter body is covered by an outer jacket. In some embodiments, the outer jacket may be coated with a lubricious material.

In some embodiments, a proximal section of the tube has less axial flexibility than a distal section of the catheter frame.

In some embodiments, the liner comprises one or more polymer layers. The one or more polymer layers may form a tubular structure. In some embodiments, the one or more layers of the liner comprises one or more of polytetrafluoroethylene (PTFE), ethylene tetrafluoroethylene (ETFE), fluorinated ethylene propylene (FEP), polyoxymethylene, polyether block ester, polyurethane, polypropylene, polyvinylchloride, polyether-ester, ether or ester based copolymers, phthalate and/or other polyester elastomers, polyamide, elastomeric polyamides, block polyamide/ethers, polyether block amide, ethylene vinyl acetate copolymers (EVA), silicones, polyethylene (PE), Marlex high-density polyethylene, Marlex low-density polyethylene, linear low density polyethylene, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR), polysulfone, nylon, nylon-12, perfluoro(propyl vinyl ether) (PFA), ethylene vinyl alcohol, polyolefin, polystyrene, epoxy, polyvinylidene chloride (PVdC), poly(styrene-b-isobutylene-b-styrene), polycarbonates, ionomers, biocompatible polymers. In some embodiments, the one or more layers of the liner comprises mixtures, combinations, copolymers, polymer/metal composites of any of the aforementioned materials. In some embodiments, the liner comprises a liquid crystal polymer (LCP).

In some embodiments, the liner may be disposed within the lumen of the tube and extend from the proximal end of the catheter body to the distal end of the catheter body. In some embodiments, the length of the liner is longer than the length of the catheter body.

In some embodiments, the liner may be partially and/or intermittently bonded or adhered to the catheter body. to further contribute to the overall flexibility of the catheter. In some embodiments, the liner is bonded to the catheter body by heat fusing/melting, adhesives, or other bonding processes. In some embodiments, a strike layer is used to assist improve bonding/attachment between the liner and the catheter body. For example, in a device utilizing a liner constructed from PTFE, a strike layer may comprise a PEBAX® powder coating between the PTFE liner and the catheter body. In some embodiments, bonding the liner to the catheter body comprises heating the catheter body at a temperature sufficient to melt the strike layer, but at a temperature lower than the temperature required to melt the liner. This may allow the melted strike layer to bond the liner to the catheter body. In some embodiments, the strike layer is a tacky material, and the liner is a more rigid material. In some embodiments, the tacky strike layer is configured to fill in the void space of the cuts for a cut tube. The tacky material may be configured to expand and compress with the cuts of the cut tube while bending.

In some embodiments, an outer jacket may comprise nylon, polyether block amide, PTFE, FEP, PFA, PET, PEEK, or combinations thereof. In some embodiments, the outer jacket has a thickness of at least about 0.0005 inches. In some embodiments, the outer jacket has a thickness of at most about 0,010 inches. In some embodiments, the strike layer has a thickness in the range of about 0.0005 inches to about 0.010 inches. In some embodiments, the outer jacket may be configured to provide an atraumatic, protective covering over the catheter body. In some embodiments, the outer jacket is configured to minimize trauma and/or pinching of tissue while advancing the catheter through torturous tissue.

In some embodiments, an outer jacket has a substantially smooth, outer surface. In some embodiments, the outer jacket may include one or more surface modifications to the outer surface. In some embodiments, the outer layer comprises the tube. The tube may comprise a cut pattern or other geometric features that alter the overall flexibility of the catheter assembly.

The outer jacket may be formed from a polymer, laminated to the liner (or strike layer enclosing the catheter body In some embodiments, the outer jacket comprises a tacky inner layer and the stiffer outer layer. In some embodiments, the inner layer and outer layer are co-extruded over the catheter body. In some embodiments, the inner layer and outer layer are simultaneously extruded. In some embodiments, the inner layer is co-extruded with the outer layer. In some embodiments, the inner layer is co-extruded with the outer layer wherein the outer layer is extruded directly behind the extrusion of the tacky inner layer. This may help control the lamination of the tacky material wherein the immediately following stiffer outer layer controls and contains the tacky material. In some embodiments, the inner layer and the outer layer are co-extruded from a single extrusion nozzle. In some embodiments, the outer layer is configured to be extruded atop the inner layer. In some embodiments, the inner layer and the outer layer are co-extruded from adjacent extrusion nozzles, wherein the outer layer extrusion nozzle directly follows the inner layer nozzle.

In some embodiments, the outer jacket comprises a polymer material. For example, nylon, polyether block amide, PTFE (polytetrafluoroethylene), FEP (fluorinated ethylene propylene), PFA (perfluoroalkoxy alkane), PET (polyethylene terephthalate) or PEEK (polyether ether ketone) In some embodiments, at least a portion of the outer layer of the outer jacket may be coated with a hydrophilic polymer coating configured to improve lubricity and/or trackability of the catheter. For example, the hydrophilic polymer coatings may comprise, polyelectrolyte and/or a non-ionic hydrophilic polymer, where the polyelectrolyte polymer can include poly(acrylamide-co-acrylic acid) salts, a poly(methacrylamide-co-acrylic acid) salts, a poly(acrylamide-co-methacrylic acid) salts, etc., and the non-ionic hydrophilic polymer may be poly(lactams), for example polyvinylpyrollidone (PVP), polyurethanes, homo-and copolymers of acrylic and methacrylic acid, polyvinyl alcohol, polyvinylethers, snapic anhydride based copolymers, polyesters,

The outer jacket may be formed by enclosing a tube wall with a co-extruded polymer of single or multiple layers. In some embodiments, assembling the catheter may comprise heat shrinking the tube or coating the tube frame via a dip coating process. The polymer jacket material may comprise nylon, polyether block amide, PTFE (polytetrafluoroethylene), FEP (fluorinated ethylene propylene), PFA (perfluoroalkoxy alkane), PET (polyethylene terephthalate) or PEEK (polyether ether ketone). Further, a portion of the catheter (or the entire length of the catheter) may be coated with a hydrophilic polymer coating to enhance lubricity and trackability. Hydrophilic polymer coatings can include, but are not limited to, polyelectrolyte and/or a non-ionic hydrophilic polymer, where the polyelectrolyte polymer can include poly(acrylamide-co-acrylic acid) salts, a poly(methacrylamide-co-acrylic acid) salts, a poly(acrylamide-co-methacrylic acid) salts, etc., and the non-ionic hydrophilic polymer may be poly(lactams), for example polyvinylpyrollidone (PVP), polyurethanes, homo-and copolymers of acrylic and methacrylic acid, polyvinyl alcohol, polyvinylethers, snapic anhydride based copolymers, polyesters, hydroxypropylcellulose, heparin, dextran, polypeptides, etc. In some embodiments, a coating to the outer jacket may be applied by a dip coating process or by spraying the coating onto the tube outer and inner surfaces. In some embodiments, the outer jacket is configured to cover the catheter body. In some embodiments, the outer jacket may be coated with a lubricious material. In some embodiments, a proximal section of the catheter body has stiffer outer jacket than a distal section of the catheter body.

In some embodiments, the outer jacket or liner may comprise a lubricious coating or film configured to improve the catheter's ability to be advanced through torturous tissue. In some embodiments, the lubricious coating may comprise silicone or hydrogel polymers or the like.

In some embodiments, manufacturing a catheter described herein comprises laminating one or more materials to a catheter body. In some embodiments, lamination comprises laminating a first layer, onto a catheter body or mandrel. In some embodiments, laminating the first layer onto the catheter body comprises extruding a first material onto the catheter body. In some embodiments, the first material comprises a soft material such as a thermoplastic elastomer, silicone, polyurethane, or other biocompatible materials.

In some embodiments, lamination comprises laminating a second layer, on top of the first layer. In some embodiments, laminating the second layer on top of the first layer comprises extruding a second material onto the first material. In some embodiments, the second material comprises of a stiffer material than the first material. In some embodiments, the second stiffer material may comprise a biocompatible polymer. Inn some embodiments, the second layer is configured to provide structural integrity and kink resistance to the catheter. This may improve the catheter's ability to navigate the tortuous body lumens without buckling, twisting, or bending.

This two-step extrusion process, involving lamination of two distinct material layers, results in a catheter possessing both softness for patient comfort and stiffness for operational efficiency. Extrusion lamination offers advantages over conventional coating or dipping methods, providing uniform material layers and improved bonding between layers. In some embodiments, the lamination of the catheter body may be tailored to achieve desired layer thicknesses and flexibility/stiffness properties, to the specific needs of various catheter applications.

In some embodiments, laminating the catheter body comprises extruding a flowable material over the catheter body. In some embodiments, the flowable material is only flowable under a particular heat and/or pressure. The flowable material may be flowed over the catheter body while in a particular environment configured to allow the material to flow, wherein the material becomes solid after the catheter body is removed from the environment.

In some embodiments, laminating the catheter body comprises laminating a cut catheter body. In some embodiments, the cut catheter body is stretched prior to lamination of the catheter body. Stretching the catheter body may increase the kerf width of one or more cuts of the cut catheter body. Stretching the catheter body may increase the flexibility of the catheter body. In some embodiments, the catheter body is stretched to a desired length and/or flexibility. In some embodiments, the catheter body is stretched to a desired degree of stretch. In some embodiments, the first and second layers are laminated to the catheter body while the catheter body is being stretched. In some embodiments, the stretched catheter body is held to a desired degree of stretch while the first and second layers are laminated to the catheter body. In some embodiments, laminating the first and second layers to the stretched catheter fixes the degree of stretch for the laminated portion of the catheter. In some embodiments, the stretched catheter body is actively stretched while the first and second layers are laminated to the catheter body. This may allow the stretch at the first end of the catheter to be less than the stretch at the second end of the catheter. In some embodiments, the first end of the catheter is less flexible than the second end of the catheter. In some embodiments, lamination the catheter body comprises laminating a first section of the stretched catheter having a first degree of stretch to fix the degree of stretch of the first section. After the first section is fixed, increase or decrease the degree of stretch of an unlaminated second portion of the stretched catheter and laminate the second section to fix the degree of stretch of the second section. This may allow for the different sections of the catheter to have different flexibility and/or stiffness properties.

### Connection Joint

In some embodiments, the catheter body comprises a single material. In some embodiments, the catheter body is formed from a single tube. In some embodiments, the catheter body is formed from a hypotube. In some embodiments, the catheter body has a consistent diameter along the length of the catheter body. In some embodiments, the catheter body has a first section comprising a first diameter and a second section comprising a second diameter. In some embodiments, the first section is a proximal and the second section is a distal section. In some embodiments, the first diameter is larger than the second diameter. In some embodiments, the first diameter is smaller than the second diameter. In some embodiments, the catheter body has a variable diameter along the length of the catheter body. In some embodiments, the catheter body has one or more transitioning sections between a first diameter and a second diameter In some embodiments, the catheter body has a one or more transition section between one or more changes in the diameter of the catheter body.

In some embodiments, the lumen of the catheter has a consistent diameter along the length of the catheter. In some embodiments, the lumen has a first section comprising a first diameter and a second section comprising a second diameter. In some embodiments, the first section is a proximal and the second section is a distal section. In some embodiments, the first diameter is larger than the second diameter. In some embodiments, the first diameter is smaller than the second diameter. In some embodiments, the catheter lumen has a variable diameter along the length of the catheter. In some embodiments, the lumen has one or more transitioning sections between a first diameter and a second diameter. In some embodiments, the lumen has a one or more transition section between one or more changes in the diameter of the catheter. In some embodiments, the diameter of the catheter body does not change with a change in the lumen diameter. In some embodiments, the diameter of the catheter body increases when the lumen diameter increases. In some embodiments, the diameter of the catheter body decreases when the lumen diameter decreases.

In some embodiments, the lumen of the catheter body is between about 0.01 inches (in) to about 0.20 in. In some embodiments, the lumen of the catheter body is between about 0.01 inches (in) to about 0.10 in. In some embodiments, the lumen of the catheter body is smaller than 0.10 in. In some embodiments, the lumen of the catheter body is larger than 0.01 in. In some embodiments, the lumen of the catheter body is at least about 0.01 in, about 0.02 in, about 0.03 in, about 0.04 in, about 0.05 in, about 0.06 in, about 0.07 in, about 0.08 in, about 0.09 in, about 0.10 in, about 0.11 in, about 0.12 in, about 0.13 in, about 0.14 in, about 0.15 in, about 0.16 in, about 0.17 in, about 0.18 in, about 0.19 in, or about 0.20 in. In some embodiments, the lumen of the catheter body is at most about 0,01 in, about 0.02 in, about 0.03 in, about 0.04 in, about 0.05 in, about 0.06 in, about 0.07 in, about 0.08 in, about 0.09 in, about 0.10 in, about 0.11 in, about 0.12 in, about 0.13 in, about 0.14 in, about 0.15 in, about 0.16 in, about 0.17 in, about 0.18 in, about 0.19 in, or about 0.20 in. In some embodiments, the catheter has a diameter in the range of about 1 French (Fr) from about 20 Fr. In some embodiments, the catheter has a diameter in the range of about 1 Fr to about 10 Fr. In some embodiments, the catheter has a diameter of about 1.0 Fr, 1.5 Fr, 2.0 Fr, 2.5 Fr, 3.0 Fr, 3.5 Fr, 4.0 Fr, 4.5 Fr, 5.0 Fr, 5.5 Fr, 6.0 Fr, 6.5 Fr, 7.0 Fr, 7.5 Fr, 8.0 Fr, 8.5 Fr, 9.0 Fr, 9.5 Fr, 10.0 Fr, 10.5 Fr, 11.0Fr, 11.5 Fr, 12.0 Fr, 12.5 Fr, 13.0 Fr, 13.5 Fr, 14.0 Fr, 14.5 Fr, 15.0 Fr, 15.5 Fr, 16.0 Fr, 16.5 Fr, 17.0Fr, 17.5 Fr, 18.0 Fr, 18.5 Fr, 19.0 Fr, 19.5 Fr, or 20.0 Fr.

In some embodiments, a catheter as described here is configured to be a variable stiffness catheter. In some embodiments, the variable stiffness catheter comprises at least a first catheter body and a second catheter body. In some embodiments, the first catheter body and the second catheter body may be releasably coupled. In some embodiments, the first catheter body and the second catheter body may be fixedly coupled. In some embodiments, the first catheter body and the second catheter body are coupled to form a single tube structure. In some embodiments, the first catheter body and the second catheter body are coupled at a connection joint. In some embodiments, the second catheter body is proximal to the first catheter body. In some embodiments, the proximal second catheter body is stiffer than the distal first catheter body. In some embodiments, the proximal second catheter body comprises stainless steel. In some embodiments, the distal first catheter body comprises NiTi.

Sometimes, the cost to manufacture may be a significant challenge to adopting cut tubes, especially laser cut tubes, in catheters. As such, the material for a cut tube should be chosen carefully, based on the intended function of the catheter. In some cases, using a cut tube that is stainless steel for its entire length is a good choice due to its relatively low cost. In cases where the cut tube structure needs to deform more than what stainless steel can safely provide, it may be better to use superelastic NiTi alloy due to its larger range of superelastic deformation. While superelastic NiTi has good superelastic deformation properties, superelastic NiTi may be expensive. In case cases, such catheter having joined catheter bodies strikes a balance between cost and performance by forming a hybrid catheter body.

In some embodiments, a variable stiffness catheter comprises a hybrid catheter body. The hybrid catheter body may combine two or more catheter bodies. In some embodiments, the two or more catheter bodies are made of different materials. In some embodiment, a catheter body may comprise a first catheter body and a second catheter body. In some embodiments, the first catheter body may comprise stainless steel. The first catheter body may be used when high levels of deformation are not expected in that region. In some embodiments, the distal end of the second catheter body comprises a superelastic NiTi. In some embodiments, the second catheter body may be used when high levels of deformation are expected.

In some embodiments, a hybrid catheter body comprises a first catheter body and a second catheter body coupled at connection joint. In some embodiments, the first catheter body is formed from a first material. In some embodiments, the second catheter body is formed from a second material. In some embodiments, the second catheter body is stiffer than the first catheter body. In some embodiments, the second catheter body comprises stainless steel In some embodiments, the first catheter body comprises NiTi. In some embodiments, the second catheter body is coupled to the first catheter body by a joint connection section. In some embodiments, it is beneficial to minimize the length of the joint connection section of the catheter

In some embodiments, the stiffness of the second catheter body is variable along the length of at least a portion of the second catheter body. In some embodiments, a proximal end of the second catheter body is stiffer than the distal end of the second catheter body. In some embodiments, the flexibility of the second catheter body is variable along a length of the second catheter body. In some embodiments, a distal end of the second catheter body is more flexible than a proximal end of the second catheter body. In some embodiments, a stiffness of the first catheter body is variable along a length of at least a portion the first catheter body. In some embodiments, a proximal end of the first catheter body is stiffer than a distal end of the first catheter body. In some embodiments, the flexibility of the first catheter body is variable along a length of the first catheter body. In some embodiments, a distal end of the first catheter body is more flexible than a proximal end of the first catheter body. In some embodiments, a proximal section of the catheter body has less axial flexibility than a distal section of the catheter body.

In some embodiments, a catheter body comprises a length from about 80 centimeters (cm) to about 200 cm. In some embodiment, the catheter body comprises a length from about 80 cm to about 200 cm, or about 90 cm to about 200 cm, about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiment, the catheter body comprises a length of about 120 cm, about 130 cm, about 140 cm, about 150 cm, about 160 cm, about 170 cm, about 180 cm, about 190 cm, or about 200 cm. In some embodiment, the catheter body comprises a length of at least about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiment, the catheter body comprises a length at most about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm.

In some embodiments, a catheter body may be a hybrid catheter. In some embodiments, the hybrid catheter comprises one or more catheter bodies. The one or more catheter bodies may each or in combination, comprises a length from about 10 centimeters (cm) to about 200 cm. In some embodiments, one or more of the catheter bodies comprise a length from about 10 centimeters (cm) to about 200 cm In some embodiments, one or more of the catheter bodies comprise a length from about 10 cm to about 200 cm, about 20 cm to about 200 cm, about 20 cm to about 200 cm, about 20 cm to about 200 cm, about 40 cm to about 200 cm, about 50 cm to about 200 cm, about 60 cm to about 200 cm, about 70 cm to about 200 cm, about 80 cm to about 200 cm, about 90 cm to about 200 cm, or about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiments, one or more of the catheter bodies comprise a length of about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, one or more of the catheter bodies comprise a length of at least about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiments, one or more of the catheter bodies comprise a length at most about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, at least one of the one or more catheter bodies comprise stainless steel. In some embodiments, at least one of the one or more catheter bodies comprise NiTi. In some embodiments, one or more of the catheter bodies comprise a first catheter body and a second catheter body.

In some embodiments, a first catheter body comprises a length from about 10 centimeters (cm) to about 200 cm. In some embodiment, a first catheter body comprises a length from about 10 cm to about 200 cm, about 20 cm to about 200 cm, about 20 cm to about 200 cm, about 20 cm to about 200 cm, about 40 cm to about 200 cm, about 50 cm to about 200 cm, about 60 cm to about 200 cm, about 70 cm to about 200 cm, about 80 cm to about 200 cm, about 90 cm to about 200 cm, about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiment, a first catheter body comprises a length of about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiment, a first catheter body comprises a length of at least about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiment, a first catheter body comprises a length at most about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, the first catheter body comprises stainless steel. In some embodiments, the first catheter body comprises NiTi.

In some embodiments, a second catheter body comprises a length from about 10 centimeters (cm) to about 200 cm. In some embodiment, a second catheter body comprises a length from about 10 cm to about 200 cm, about 20 cm to about 200 cm, about 20 cm to about 200 cm, about 20 cm to about 200 cm, about 40 cm to about 200 cm, about 50 cm to about 200 cm, about 60 cm to about 200 cm, about 70 cm to about 200 cm, about 80 cm to about 200 cm, about 90 cm to about 200 cm, about 100 cm to about 200 cm, about 110 cm to about 200 cm, about 120 cm to about 200 cm, about 130 cm to about 200 cm, about 140 cm to about 200 cm, about 150 cm to about 200 cm, about 160 cm to about 200 cm, about 170 cm to about 200 cm, about 180 cm to about 200 cm, or about 190 cm to about 200 cm. In some embodiment, a second catheter body comprises a length of about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiment, a second catheter body comprises a length of at least about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, or about 190 cm. In some embodiment, a second catheter body comprises a length at most about 10 cm, about 20 cm, about 30 cm, about 40 cm, about 50 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm. In some embodiments, the second catheter body comprises stainless steel. In some embodiments, the second catheter body comprises NiTi.

In some embodiments, the joint connection section may comprise a proximal joint connection section coupled to the first catheter body and a distal joint connection section coupled to the second catheter body. In some embodiments, the second catheter body and the distal joint connection section are formed from a single material. In some embodiments, the first catheter body and the proximal joint connection section are formed from a single material. In some embodiments, the joint connection section is formed using a cutting element. In some embodiments, the joint connection section comprises one or more joint features, configured to couple the second catheter body to the first catheter body. In some embodiments, the joint feature may comprise a receiver element and an insertion element, wherein the receiver element is configured to receive and securely hold the insertion element forming a joint. In some embodiments, the joint feature may comprise a pair of prongs configured to receive and securely hold a post. The post may be configured to form a snap fit pair junction with the pair of prongs.

The post may comprise a flared tip configured to engage with teeth of the pair of prongs once inserted, such that the posts cannot be pulled out of the joint. In some embodiments, a welding pad extends between the pair of prongs. In some embodiments, the welding pad may be configured to extend a distance between the prongs such that the tip of the welding pad is adjacent to and/or in contact with the flared tip of the post while the post is engaged with the prongs. In some embodiments, the post is bonded to the welding pad. The post may be bonded to the welding pad using welding, adhesive, soldering or a combination thereof. In some embodiments, the joint feature is configured so that the post and the prongs can be snapped together quickly and accurately during assembly to reduce assembly cost and improve quality of the finished catheter. To ensure permanent fixation of the first and second catheter bodies, a laser weld, solder, or other joining techniques can be used to ensure the first and second catheter bodies stay attached to each other during use. In some embodiments, the length of the second catheter body is greater than the length of the first catheter body. In some embodiments, the length of the second catheter body is less than the length of the first catheter body. In some embodiments, the length of the second catheter body is equal to the length of the first catheter body.

In some embodiments, the joint connection section comprises an inner liner. In some embodiments, the joint connection section comprises an outer jacket. In some embodiments, the joint connection section does not have one or more of the inner liner or the outer jacket. In some embodiments, the connection section does not comprise a cut filler. In some embodiments, the connection section comprises a cut filler. In some embodiments, the lamination of the catheter layers may be configured to form an additional coupling element of the joint connection section. In some embodiments, the joint connection section comprise three coupling forces configured to prevent movement and/or separation of the second catheter body and first catheter body, 1) The engagement between the receiver (e.g., a pair of prongs) and the insertion element (e.g., a post); 2) the bonding element coupling the welding pad and the tip of the post (i.e., welded connection, soldered connection); and 3) the lamination of the catheter body at the joint connection section. In some embodiments, the catheter body may comprise more than two catheter bodies coupled together. In some embodiments, adjacent catheter bodies are coupled by a connection joint. In some embodiments, a first pair of catheter bodies are couple using a first connection joint and a second pair of catheter bodies are couple using a second connection joint. In some embodiments, the first connection joint has a first configuration, and the second connection joint has a second configuration. In some embodiments, the first connection joint connection is different than the second connection joint configuration. In some embodiments, the first connection point is proximal the second connection joint. In some embodiments, the first connection joint has a length shorter than the length of the second connection joint. In some embodiments, the first connection joint has a length longer than the length of the second connection joint. In some embodiments, the length of a connection joints between catheter bodies is equal to or less than a proximal connection joint.

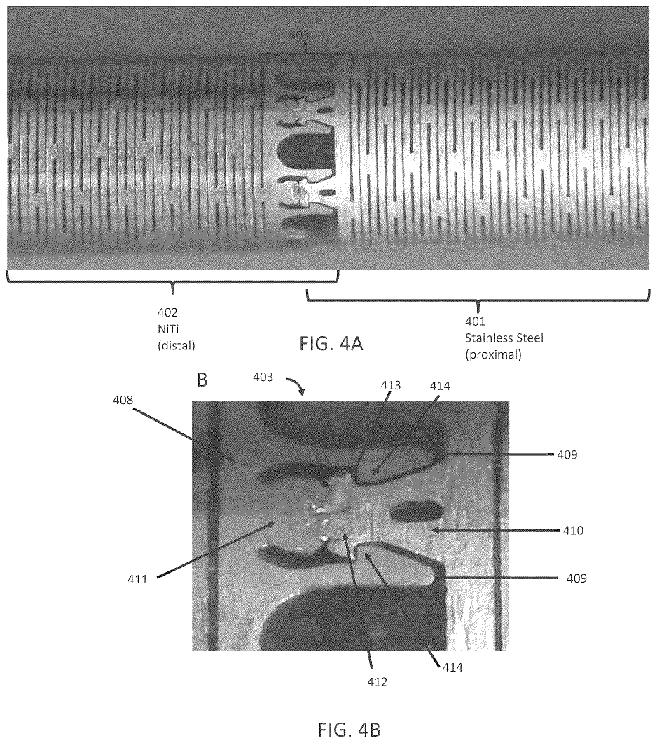

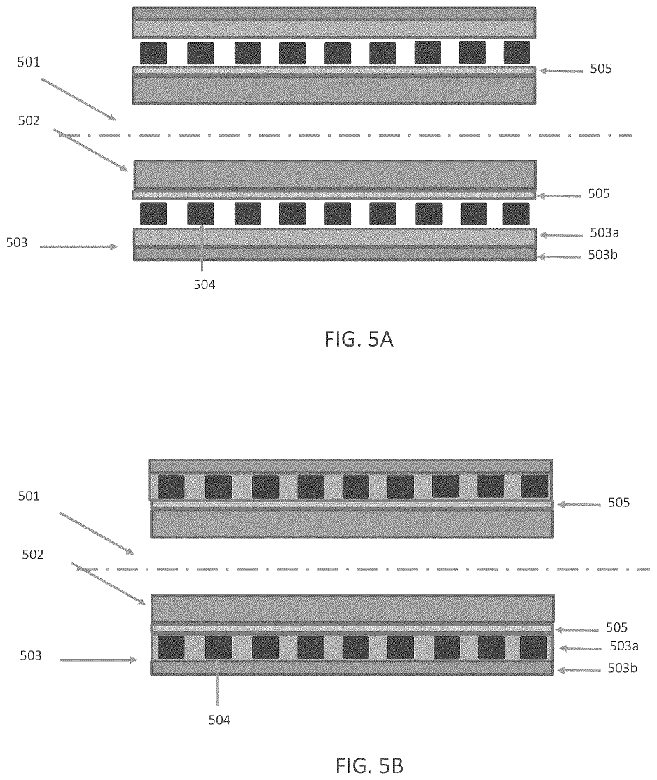

FIGS. 4A-4B illustrates an embodiment of a variable stiffness catheter having a first catheter body and a second catheter body that are joined. In comes cases, such catheter having joined catheter bodies strikes a balance between cost and performance by forming a hybrid catheter body. In some embodiments, the hybrid catheter body may combine two catheter bodies made of different materials. In some embodiment, a catheter body may comprise a first catheter body 401 and a second catheter body 402. In some embodiments, the first catheter body may comprise stainless steel The first catheter body may be used when high levels of deformation are not expected in that region. In some embodiments, the distal end of the second catheter body 402 comprises a superelastic NiTi. In some embodiments, the second catheter body may be used when high levels of deformation are expected.

FIG. 4A illustrates an embodiment of a hybrid catheter body. In some embodiments, a catheter body comprises a first catheter body 401 and a second catheter body 402 coupled at connection joint. In some embodiments, the second catheter body is formed from a first material. In some embodiments, the first catheter body is formed from a second material. In some embodiments, the second catheter body is stiffer than the first catheter body. In some embodiments, the second catheter body comprises stainless steel. In some embodiments, the first catheter body comprises NiTi. In some embodiments, the second catheter body is coupled to the first catheter body by a joint connection section 403. In some embodiments, it is beneficial to minimize the length of the joint connection section 403 of the catheter.

In some embodiments, the stiffness of the second catheter body is variable along the length of the first section. In some embodiments, a proximal end of the second catheter body is stiffer than the distal end of the second catheter body. In some embodiments, the flexibility of the second catheter body is variable along a length of the second catheter body. In some embodiments, a distal end of the second catheter body is more flexible than a proximal end of the second catheter body. In some embodiments, the stiffness of the first catheter body is variable along a length of the distal section. In some embodiments, a proximal end of the first catheter body is stiffer than a distal end of the first catheter body In some embodiments, the flexibility of the first catheter body is variable along a length of the first catheter body. In some embodiments, a distal end of the first catheter body is more flexible than a proximal end of the first catheter body. In some embodiments, a proximal section of the catheter body has less axial flexibility than a distal section of the catheter body.

In some embodiments, the joint connection section 403 may comprise a proximal joint connection section coupled to the first catheter body and a distal joint connection section coupled to the second catheter body. In some embodiments, the second catheter body and the distal joint connection section are formed from a single material. In some embodiments, the first catheter body and the proximal joint connection section are formed from a single material. In some embodiments, the joint connection section is formed using laser cutting. In some embodiments, the joint connection section comprises one or more joint features 408, configured to couple the second catheter body to the first catheter body. FIG. 4B illustrates an example of a joint feature configuration for coupling the second catheter body and the first catheter body. In some embodiments, the joint feature 408 may comprise a receiver element and an insertion element, wherein the receiver element is configured to receive and securely hold the insertion element forming a joint In some embodiments, the joint feature 408 may comprise a pair of prongs 409 configured to receive and securely hold a post 410. The post 410 may be configured to form a snap fit pair junction with the pair of prongs 409. The post 410 may comprise a flared tip 413 configured to engage with teeth 414 of the pair of prongs once inserted, such that the posts cannot be pulled out of the joint. In some embodiments, a welding pad 411 extends between the pair of prongs. In some embodiments, the welding pad may be configured to extend a distance between the prongs 409 such that the tip of the welding pad is adjacent to and/or in contact with the flared tip 413 of the post 410 while the post is engaged with the prongs 409. In some embodiments, the post 410 is bonded to the welding pad 411. The post 410 may be bonded to the welding pad 411 using welding, adhesive, soldering or a combination thereof. In some embodiments, the joint feature 408 is configured so that the post 410 and the prongs 409 can be snapped together quickly and accurately during assembly to reduce assembly cost and improve quality of the finished catheter. To ensure permanent fixation of the first and second catheter bodies, a laser weld 412, solder, or other joining techniques can be used to ensure the first and second catheter bodies stay attached to each other during use. In some embodiments, the length of the second catheter body is greater than the length of the first catheter body In some embodiments, the length of the second catheter body is less than the length of the first catheter body. In some embodiments, the length of the second catheter body is equal to the length of the first catheter body.

In some embodiments, the joint connection section comprises an inner liner. In some embodiments, the joint connection section comprises an outer jacket. In some embodiments, the joint connection section does not have one or more of the inner liner or the outer jacket. In some embodiments, the connection section does not comprise a cut filler. In some embodiments, the connection section comprises a cut filler. In some embodiments, the lamination of the catheter layers may be configured to form an additional coupling element of the joint connection section. In some embodiments, the joint connection section comprise three coupling forces configured to prevent movement and/or separation of the second catheter body and first catheter body, 1) The engagement between the receiver (e.g., the pair of prongs 409) and the insertion element (e.g., the post 410); 2) the bonding element coupling the welding pad 411 and the tip of the post 410 (i.e., welded connection, soldered connection); and 3) the lamination of the catheter body at the joint connection section. In some embodiments, the catheter body may comprise more than two catheter bodies coupled together.

In some embodiments the joint connection section comprises a length from about 0.10 centimeters (cm) to about 5.0 cm. In some embodiment, a second catheter body comprises a length from about 0.50 cm to about 5.0 cm, about 1.0 cm to about 5.0 cm, about 1.5 cm to about 5.0 cm, about 2.0 cm to about 5.0 cm, about 2.50 cm to about 5.0 cm, about 3.0 cm to about 5.0 cm, about 3.5 cm to about 5.0 cm, about 4.0 cm to about 5.0 cm, or about 4.5 cm to about 5.0 cm. In some embodiments, the joint connection section comprises a length of at least about 0.5 cm, about 1.5 cm, about 2.0 cm, about 2.5 cm, about 3.0 cm, about 3.5 cm, about 4.0 cm, about 4.5 cm, or about 5.0 cm. In some embodiments, the joint connection section comprises a length of at most about 0.5 cm, about 1.5 cm, about 2.0 cm, about 2.5 cm, about 3.0 cm, about 3.5 cm, about 4.0 cm, about 4.5 cm, or about 5.0 cm.

In some embodiment, the joint connection section comprises a length from about 0.10 cm to about 2 cm, about 0.20 cm to about 2 cm, about 0.30 cm to about 2 cm, about 0.40 cm to about 2 cm, about 0.50 cm to about 2 cm, about 0.60 cm to about 2 cm, about 0.70 cm to about 2 cm, about 0.80 cm to about 2 cm, or about 0.90 cm to about 2.0 cm, about 1.0 cm to about 2.0 cm, about 1.1 cm to about 2.0 cm, about 1.20 cm to about 2.00 cm, about 1.30 cm to about 2.0 cm, about 1.40 cm to about 2.0 cm, about 1.50 cm to about 2.0 cm, about 1.60 cm to about 2.0 cm, about 1.70 cm to about 2.0 cm, about 1.90 cm to about 2.0 cm, or about 1.90 cm to about 2.0 cm. In some embodiments, the joint connection section comprises a length of about 0.10 cm, about 0.20 cm, about 0.30 cm, about 0.40 cm, about 0.50 cm, about 0.60 cm, about 0.70 cm, about 0.80 cm, about 0.90 cm, about 1.0 cm, about 1.10 cm, about 1.20 cm, about 1.30 cm, about 1.40 cm, about 1.50 cm, about 1.60 cm, about 1.70 cm, about 1.80 cm, about 1.90 cm, or about 2.0 cm. In some embodiments, the joint connection section comprises a length of at least about 0.10 cm, about 0.20 cm, about 0.30 cm, about 0.40 cm, about 0.50 cm, about 0.60 cm, about 0.70 cm, about 0.80 cm, about 0.90 cm, about 1.0 cm, about 1.10 cm, about 1.20 cm, about 1.30 cm, about 1.40 cm, about 1.50 cm, about 1.60 cm, about 1.70 cm, about 1.80 cm, about 1.90 cm, or about 2.0 cm. In some embodiments, the joint connection section comprises a length at most about 0.10 cm, about 0.20 cm, about 0.30 cm, about 0,40 cm, about 0.50 cm, about 0.60 cm, about 0.70 cm, about 0.80 cm, about 0.90 cm, about 1.0 cm, about 1.10 cm, about 1.20 cm, about 1.30 cm, about 1.40 cm, about 1.50 cm, about 1.60 cm, about 1.70 cm, about 1.80 cm, about 1.90 cm, or about 2.0 cm. In some embodiments, the joint connection section comprises stainless steel, NiTi, or a combination thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element is present or not (for example, detection). These terms can include quantitative, qualitative, or quantitative and qualitative determinations. Assessing is alternatively relative or absolute. “Detecting the presence of” includes determining the amount of something present, as well as determining whether it is present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be endometriosis. In some cases, the subject is not necessarily diagnosed or suspected of being at high risk for the disease.

The term “in vivo”is used to describe an event that takes place in a subject's body.

The term “ex vivo” is used to describe an event that takes place outside of a subject's body. An “ex vivo” assay is not performed on a subject Rather, it is performed upon a sample separate from a subject. An example of an “ex vivo”assay performed on a sample is an “in vitro”assay.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

In some embodiments, flexibility refers to the ability of a material to deform elastically and return to its original shape when the applied stress is removed. In some embodiments, flexibility refers to the ability of a material to deform or bend without breaking. In some embodiments, a more flexible material may allow for a material to navigate through curved or tortuous pathways. In some embodiments, flexibility may be measured in terms of the catheter's bending radius, ability to recover its original form after deformation, and/or resistance to kinking under various conditions. In some embodiments, the flexibility may be measured as yield strength, elastic modulus, and/or flexural modulus. In some embodiments, the flexibility may be measured by a durometer.

In some embodiments, stiffness refers to the extent to which a material resists deformation in response to an applied force. In some embodiments, stiffness may refer to hardness of the material. In some embodiments, the flexibility may be measured as elastic modulus, Young's modulus, and/or tensile modulus. In some embodiments, stiffness is calculated as a ratio of force to deflection.

The section headings used herein are for organizational purposes only and are not to be construed as limiting the subject matter described.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Numbered Embodiments

Provided herein are following exemplary embodiments:

1. A catheter comprising:
a catheter body comprising
a first section comprising a plurality of first cuts;
a second section adjacent to the first section, the second section comprising a plurality of second cuts; and
a cut filler comprising a polymer that is filling at least one of a first cut of the plurality of the first cut and a second cut of the plurality of second cuts,
wherein the first cut of the plurality of first cuts has an average first kerf that is wider than an average kerf of the second cut of the plurality of second cuts.

2. The catheter of embodiment 1, wherein the catheter comprises an outer jacket covering at least an outer surface of the catheter body.

3. The catheter of embodiment 1 or 2, wherein the catheter comprises a liner covering at least a portion of a lumen of the catheter body

4. The catheter of embodiment 3, wherein the liner comprises a polymer having a low coefficient of friction.

5. The catheter of embodiment 3, wherein the liner comprises a polymer, wherein a polymer comprises polyethylene, PTFE, or poly(ether-block-amide), or a combination thereof.

6. The catheter of any one of preceding embodiments, wherein the cut filler contacts the outer jacket.

7. The catheter of any one of preceding embodiments, wherein the cut filler is bonded to the outer jacket.

8. The catheter of any one of preceding embodiments, wherein the cut filler contacts the liner.

9. The catheter of any one of preceding embodiments, wherein the cut filler is bonded to the liner.

10. The catheter of embodiment 9, wherein the liner comprises a strike layer to facilitate bonding of the liner to the cut filler.

11. The catheter of embodiment 9, wherein a surface of the finer is modified to facilitate bonding of the liner to the cut filler.

12. The catheter of embodiment 1, wherein the modification comprises at least one of chemical modification, textural modification, mechanical modification, or plasma modification.

13. The catheter of any one of preceding embodiments, wherein at least one of the plurality of first cuts with a plurality of first cuts is in an interrupted spiral pattern.

14. The catheter of any one of preceding embodiments, wherein the first section is distal to the second section.

15. The catheter of any one of preceding embodiments, wherein the second section is stiffer than the first section.

16. The catheter of any one of preceding embodiments, wherein the first section is more flexible than the second section.

17. The catheter of any one of preceding embodiments, wherein a stiffness of the first section is variable along the length of the first section.

18. The catheter of embodiment 17, wherein a proximal end of the first section is stiffer than a distal end of the first section.

19. The catheter of any one of preceding embodiments, wherein a flexibility of the first section is variable along a length of the first section.

20. The catheter of embodiment 19, wherein a distal end of the first section is more flexible than a proximal end of the first section.

21. The catheter of any one of preceding embodiments, wherein a stiffness of the second section is variable along a length of the second section.

22. The catheter of embodiment 21, wherein a proximal end of the second section is stiffer than a distal end of the second section.

23. The catheter of any one of preceding embodiments, wherein a flexibility of the second section is variable along a length of the second section.

24. The catheter of embodiment 23, wherein a distal end of the second section is more flexible than a proximal end of the second section.

25. The catheter of any one of preceding embodiments, wherein the plurality of first cuts are not parallel to each other.

26. The catheter of any one of preceding embodiments, wherein the plurality of second cuts are not parallel to each other.

27 The catheter of any one of preceding embodiments, wherein the catheter body comprises a metal.

28. The catheter of embodiment 27, wherein the metal comprises stainless steel, or nitinol, or a combination thereof.

29. The catheter of any one of preceding embodiments, wherein the catheter body comprises a radiopaque material

30. The catheter of embodiment 29, wherein the radiopaque material comprises tungsten, platinum, iridium, or a combination thereof.

31. The catheter of any one of preceding embodiments, wherein the catheter body comprises a polymer.

32. The catheter of embodiment 31, wherein the polymer comprises a liquid crystal polymer (LCP), polyimide, PEEK, or nylon, or a combination thereof.

33. The catheter of any one of preceding embodiments, wherein the catheter comprises a reinforcement.

34. The catheter of embodiment 33, wherein the reinforcement comprises a braid.

35. The catheter of any one of preceding embodiments, wherein at least one of the plurality of first cuts and the plurality of second cuts is prepared by laser cutting.

36. The catheter of any one of preceding embodiments, wherein the plurality of first cuts bas a first pitch and the plurality of the second cuts has a second pitch.

37. The catheter of embodiment 36, wherein the first pitch is the same as the second pitch.

38. The catheter of embodiment 36, wherein the first pitch is greater than the second pitch.

39. The catheter of any one of preceding embodiments, wherein the plurality of first cuts bas a plurality of first bridges having a first kerf and the plurality of the second cuts has a plurality of second bridges having a second kerf.

40. The catheter of embodiment 39, wherein the first kerf is the same as the second kerf

41. The catheter of embodiment 39, wherein the first kerf is greater than the second kerf.

42. The catheter of any one of preceding embodiments, wherein an average first kerf of the first cut ranges from 0.0001 inch to 0.01 inch.

43. The catheter of any one of preceding embodiments, wherein an average second kerf of the second cut ranges from 0.0001 inch to 0.01 inch.

44. The catheter of any one of preceding embodiments, wherein a kerf of the first cut varies along a radial direction of the first cut.

45. The catheter of any one of preceding embodiments, wherein a kerf of the second cut varies along a radial direction of the second cut.

46. The catheter of any one of preceding embodiments, wherein the catheter body comprises a connection joint at a distal end of the catheter body or a proximal end of the catheter body or both ends of the catheter body.

47. The catheter of embodiment 46, wherein the connection joint comprises a snap or a post or a combination thereof.

48. The catheter of embodiment 46 or 47, wherein the connection joint is configured to connect the catheter to a connection joint of a second catheter.

49. The catheter of embodiment 48, wherein the connection joint of the catheter body and the connection joint of the second catheter are permanently fixed to each other.

50. The catheter of any one of embodiments 46 to 49, wherein the catheter comprises a first material different from the second material comprising the second catheter.

51. A method of preparing a catheter of any one of the preceding embodiments, the method comprising:
(a) applying a plurality of cuts to a tube to form a cut tube having a plurality of cuts;
(b) applying a first stretch to a first section of the cut tube, wherein the first stretch increases an average kerf of the plurality of cuts in the first section from a first initial kerf to a first final kerf;
(c) applying a second stretch to a second section of the cut tube proximal to the first section, wherein the second stretch increases an average kerf of the plurality of cuts in the second section from a second initial kerf to a second final kerf, wherein the first final kerf is larger than the second final kerf; and
(d) laminating a portion of the stretched cut tube with a polymer sheet comprising a first layer of a first polymer and a second layer of a second polymer, wherein the first polymer is more elastic than the second polymer, wherein the first polymer fills the plurality of cuts in the portion of the stretched cut tube.

52. The method of embodiment 51, wherein the plurality of cuts is applied by laser cutting.

53. The method of embodiment 51 or 52, wherein the second polymer allows for easy handling of the first polymer.

54. The method of any one of preceding embodiments, wherein the method further comprises lining a portion of a lumen of the cut tube with a liner polymer.

55. The method of embodiment 54, wherein the liner polymer contacts the first polymer filling the plurality of cuts.

1. A catheter comprising:
a catheter body comprising
a first section comprising a plurality of first cuts, wherein the plurality of first cuts comprises a plurality of first interrupted spirals and has an average first kerf;
a second section adjacent to the first section, the second section comprising a plurality of second cuts, wherein the plurality of second cuts comprises a plurality of second interrupted spirals and has an average second kerf, and wherein the average first kerf is wider than the average second kerf, and
a cut filler comprising a polymer that is filling at least one of the plurality of the first cuts and the plurality of second cuts;
an outer jacket covering at least an outer surface of the catheter body; and
an inner layer is bonded to at least a portion of an inner surface of the catheter body,
wherein the inner surface defines a lumen of the catheter body, and
wherein the cut filler contacts the outer jacket and the inner layer.

2. The catheter of claim 1, wherein at least one of the plurality of first cuts and the plurality of second cuts comprise laser cuts.

3. (canceled)

4. The catheter of claim 1, wherein the inner layer comprises a strike layer to facilitate bonding of the inner layer to the cut filler.

5. (canceled)

6. The catheter of claim 1, wherein the second section is stiffer than the first section.

7. The catheter of claim 1, wherein the first section is more flexible than the second section.

8. The catheter of claim 7, wherein a flexibility of the first section is variable along a length of the first section.

9. The catheter of claim 7, wherein a flexibility of the second section is variable along a length of the second section.

10. The catheter of claim 1, wherein the plurality of first cuts are not parallel to each other.

11. The catheter of claim 1, wherein the plurality of second cuts are not parallel to each other.

12. The catheter of claim 1, wherein the catheter body comprises a metal.

13. The catheter of claim 1, wherein the catheter body comprises a radiopaque material.

14. The catheter of claim 1, wherein the plurality of first cuts has a first pitch and the plurality of the second cuts has a second pitch and wherein the first pitch is the same as the second pitch.

15. The catheter of claim 1, wherein the plurality of first cuts has a first pitch and the plurality of the second cuts has a second pitch and wherein the first pitch is greater than the second pitch.

16. The catheter of claim 1, wherein at least one of the average first kerf or the average second kerf ranges from 0.0001 inch to 0.01 inch.

17. The catheter of claim 1, wherein at least one of the first cuts or the second cuts varies in width along a radial direction of the cut.

18. The catheter of claim 1, wherein the catheter body comprises a connection joint at a distal end of the catheter body or a proximal end of the catheter body and wherein the connection joint is configured to connect the catheter to a connection joint of a second catheter.

19. The catheter of claim 1, wherein the catheter comprises a flat tip at a distal end of the catheter body.

20. (canceled)

21. (canceled)

22. The catheter of claim 1, wherein the average first kerf is formed by applying a first stretch and the average second kerf is formed by applying a second stretch and wherein the first stretch is different from the second stretch.

23. The catheter of claim 12, wherein the metal comprises stainless steel, nitinol, super elastic NiTi alloy super elastic nickel titanium, shape memory nickel titanium, TiNi, nickel titanium, NiTiHf, NiTiPd, or NiMnGa, or a combination thereof.

24. The catheter of claim 1, wherein the catheter body comprises a polymer comprising LCP (liquid crystal polymer), polyimide, PEEK, or nylon, or a combination thereof.
